SEQUENCE LISTING (18 sequences)

SEQ ID NO: 1 - DNA, 3224 bp, Mus musculus
ctatcctgaccctcgtgcttagaccattttctattctctctttgttttgtggtcgaatttcttgggagtttgtgtggaggtgacttcttctgaaatctgacagtccctctttggtactcccctgtccttttctgggaagtggtgattttggcgtccacacttcctcttccttcctgggtagcagcaacagctaacatggagtcttccttatatctcccttggtcccttggatttcctttctatcagccccagaggataccttgcaaggctgcatcagtcctcccatccctggctgttgcctctcggagaccagcagcaaaatcggagctcagcaggcctgggtctgttcagtcctgggtggggactagctttctggccttcaggaacatgtcttgacatgttcagccctggaattgaagaggtggggctgcttcagtgcagggtggtggagacctgatgatacccaactaccagctgtggggtgaggaggagcatgaatggagacagagaccccagataattaaggacgtcccactttgccagcagaataaaaggtggtatgtatcttgtgacatgtatcaggtgaaggatgcttttgtgcatctgtgtgtgtgtgcctggggtgtgtatgtgtcaggtactgtatgtagtcattttgtcactttgtcattttggggtctggagggctcctccaaccatgtttctgagtatacattcacgtgcaatggtgtgcctgactatacattcaagtgcaaggccaagaatgtttgttagaaagactgagtagtcccagacttaataaatatttgttgagtacttttgtggtgctctgggaagccagaagttgtttaaaataaatctctccaacaccagtagggtaaaggcacaggaggtcacagcactcagcagttcagtataagcctttattcaagctgttttctcccaaagtaaacagacagacaatgtcacttctatctgagaagcctggaggccaaggggatttgggcagttttgacatcctgtgctggcccctgacagcccagccctgggatggacgacttggatgcagggactggaccgttcaggagctggggcattgtgggagtggccattatgtctgtcctggtttgggggtctgaagggggtccttcaactgtgtttctgaacaggatgaggcttttgaggggggttgggaaggtggccaagcccttcccagacttccaccccccatcacagaagaggaggcctgtctaggtcagctaaatccgaggaggaagactgggcatgtctgggcagcgatctctagacatcggagagcagatgtcagaactcacagcttctccaaagcaggattttgatcttttaactaaagatatccgttcaaactaagactccagtctctgctttatttaaaatttttgtttgtgtttgttttgagagagagaggaatttgtttttgttttagaggcaggatgtcatgtaaccttgatgaattattggtcctcctgtttctgtctcctgagtgccgggattacagatgtatgccgctatcatctagatgatgccttactagggatccaactcctggcttcatacatgttaggcaagcgctatattaacggagctacatccctttttggatgcatggtgatctcagatagctcaggctagccttgagctccaaatcccccctgcctcccaagtaccgtgatttcaggcatgtacctctatgcttagctgagatggtggtcttgctatgtagcccatgtgaccaggctggatcgtgtaacaagactgaagaaaacccccttctgctgggtgtgatggctcagacctgtagtcttaactctaagcaaggaagttaaggcaggaagattgccttgaatatgagggccaccctgggctacatagcgaggcctcgtctcaaaagaccccaaacagaaggaaataaaactgactagagacatggaggaaggtgggaacggagaatgtcttactgctatgtctgtcaggaacatccgtagatgtttctagaattgtcctttatcaatgtcattttagtggactgtctggatcttgggagggggagtattagacatttccctcattttggacccagagaaagaaatctgcaagcagagtactctgggcagcttgccagaggtcagcaggtagccattagtgtggtcccagtcaggtcttgatgctctcacttgcaggatgtattatggtgtgagaaatgttcacatgctggcttctgaagaggggagaggggaggtaaggagcctggcggctgtttttcttggtagtccgtggtctgagaactggactcaatctccccgatttctgaggcggttgacagcatcctttccttctgtggaactgctttcctcgtctgtgagacagggaggaaatgatcgcgttctggacccgatgtccgaggggcttctgggaggagggggaaaaaaatctccagacatagtgggacttcttgggattttaaactattttttattatttatgggcttgttttgtttttgagagggtctcaatggatagcccaggctggtcttgaacctgtaacgcccctcgtgcctcaatctcccaagtataggattccaggcttttgctatcatactcaaatgatcaatttatttatatttgaaacagtgtcacatatttcaagttggtctccatcggaataggtagctgtcaaaacgaccttgaatgcctatttccccctcctcaccccccactgggcgctgttattacagacgtgaccccgcatgcccagtttatggggccctggagctccaacccagggcttcacttccagcaagttaggcaaacactgtaccaacagacccacctcccgaaacccaggattttatttactaatatcagtgatctggaaaagagttagtccttcccacagttggtcagggacagacccataaacactcactcagctctaactgtactgttgttcatagatgtatgtggctctgctggtgcgctgcgtgacaagagaatagcccaggtgtgggggaggggagggcgcgccctcttaacgcgcgccggttctagctgtctggcgcgggcttagattcccagaggggagggcgggccagcgagtccccgggatcggtgaaggagtgggttggtcctgcctctgagggggcggggcctgggccgacgctataaggaggcagctcgacgccaac

SEQ ID NO: 2 - protein, 660 aa, Artificial [sequence omitted]

SEQ ID NO: 3 - DNA, 2049 bp, Artificial
  synthetic oligonucleotide
atgtccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgatctcgagccatctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagtgaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacagcttgataacgcgtttaattaactcgaggttttcgaggtcgacggtatcgataagcttgatatcgaattccg

SEQ ID NO: 4 - protein, 343 aa, Artificial
  synthetic polypeptide
MSNLLTVHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLEDGD

SEQ ID NO: 5 - DNA, 1032 bp, Artificial
  synthetic oligonucleotide
atgtccaatttacttaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattag

SEQ ID NO: 6 - protein, 310 aa, Artificial
  synthetic polypeptide
MRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGLVWRSMEHPVKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEAADAHRLHAPTSRGGASVEETDQSHLATAGSTSSHSLQKYYITGEAEGFPATA

SEQ ID NO: 7 - DNA, 1020 bp, Artificial
  synthetic oligonucleotide
ctcgagccatctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagtgaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacagcttgataacgcgtttaattaactcgaggttttcgaggtcgacggtatcgataagcttgatatcgaattccg

SEQ ID NO: 8 - DNA, 574 bp, Artificial
  synthetic oligonucleotide
cccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataa

SEQ ID NO: 9 - protein, 239 aa, Artificial
  synthetic polypeptide
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 10 - DNA, 649 bp, Artificial
  synthetic oligonucleotide
tatggccacaaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagacccc

SEQ ID NO: 11 - DNA, 20 bp, Artificial
  synthetic oligonucleotide primer
accagccagctatcaactcg

SEQ ID NO: 12 - DNA, 19 bp, Artificial
  synthetic oligonucleotide primer
ttacattggtccagccacc

SEQ ID NO: 13 - DNA, 21 bp, Artificial
  synthetic oligonucleotide primer
tggaggaggacaaactggtca

SEQ ID NO: 14 - DNA, 25 bp, Artificial
  synthetic oligonucleotide primer
ttgttccctttctgcttcatcttgc

SEQ ID NO: 15 - DNA, 21 bp, Artificial
  synthetic oligonucleotide primer
atcaactaccgccacctcgac

SEQ ID NO: 16 - DNA, 25 bp, Artificial
  synthetic oligonucleotide primer
gacacgctgaacttgtggccgttta

SEQ ID NO: 17 - DNA, 25 bp, Artificial
  synthetic oligonucleotide primer
tatcatgtctggatcaattcataac

SEQ ID NO: 18 - DNA, 23 bp, Artificial
  synthetic oligonucleotide primer
tttcgacctgcagccaataagct

(19) United States
(12) Patent Application Publication
(10) Pub. No.: US 2022/0386574 A1
(43) Pub. Date: Dec. 8, 2022

(54) P21-CRE ANIMAL MODELS

(71) Applicant: Mayo Foundation for Medical Education and Research, Rochester, MN (US)

(72) Inventors: James L. Kirkland, Rochester, MN (US); Ming Xu, Avon, CT (US); Tamar Tchkonia, Rochester, MN (US)

(21) Appl. No.: 17/831,757

(22) Filed: Jun. 3, 2022

Related U.S. Application Data
(60) Provisional application No. 63/196,390, filed on Jun. 3, 2021.

(51) Int. Cl. A01K 67/027; A61K 49/00; C07K 14/435; C12N 15/62

(52) U.S. Cl. CPC A01K 67/0275; A61K 49/0008; C07K 14/435; C12N 15/62; A01K 2217/052; A01K 2217/203; A01K 2227/105; A01K 2267/03; C07K 2319/00

(57) ABSTRACT

Transgenic non-human animal models for cellular senescence are provided herein, as are methods and materials for making and using the transgenic non-human animal models. For example, a p21-Cre mouse model for cellular senescence is provided herein.

### CROSS-REFERENCE TO RELATED APPLICATIONS

This application claims the benefit of U.S. Patent Application Ser. No. 63/196,390, filed on Jun. 3, 2021. The disclosure of the prior application is considered part of (and is incorporated by reference in) the disclosure of this application.

### SEQUENCE LISTING

This application contains a Sequence Listing that has been submitted electronically as an ASCII text file named 07039-2041001_ST25.txt. The ASCII text file, created on Jun. 3, 2022, is 26.7 kilobytes in size. The material in the ASCII text file is hereby incorporated by reference in its entirety.

### TECHNICAL FIELD

This document relates to transgenic non-human animal models for cellular senescence, and to methods and materials for making and using the transgenic non-human animal models.

### BACKGROUND

Cellular senescence is a cell fate characterized by essentially irreversible proliferative arrest (Gorgoulis et al., Cell 179:813-827, 2019). A variety of stimuli, including DNA damage, dysfunctional telomeres, oncogenic proteins, fatty acids, reactive oxygen species (ROS), mitogens, and cytokines, can act alone or in combination to drive cells into the cellular senescence fate through pathways involving p16/Rb (retinoblastoma), p53/p21, and possibly other factors (Tchkonia et al., J Clin Invest 123:966-972, 2013). These stimuli can contribute to widespread changes in gene expression that underlie senescence-associated growth arrest, the senescence-associated secretory phenotype (SASP), resistance to apoptosis, and changes in morphology (Tchkonia et al., supra; and Kirkland and Tchkonia, EBioMedicine 21:21-28, 2017). In these respects, cellular senescence can be considered a state of major cellular programming in addition to differentiation, proliferation, or apoptosis. Intracellular autocrine loops reinforce progression to irreversible replicative arrest, heterochromatin formation, and initiation of the pro-inflammatory SASP over a span of days to weeks (Tchkonia et al., supra; Kirkland and Tchkonia, supra; and Kuilman, et al., Cell 133:1019-1031, 2008).

Senescent cell burden increases in various tissues with aging (Wang et al., Aging Cell 8:311-323, 2009) and multiple chronic conditions (Zhu et al., Curr Opin Clin Nutr Met Care 17:324-328, 2014). Depending on specific tissues and varied pathological states, the percent of senescent cells can range from 1-20% (Baker et al., Nature 530:184-189, 2016; Xu et al., Elife 4:e12997, 2015; and Xu et al., Proc Natl Acad Sci USA 112:E6301-6310, 2015). Even when the percentage is relatively low, senescent cells can cause substantial tissue dysfunction (Kirkland et al., J Am Geriatr Soc 65:2297-2301, 2017). Senescent cells can elicit damage in both an autocrine and paracrine fashion, such that in addition to inducing intracellular dysfunction by autocrine signaling, senescent cells can be “contagious” and induce cellular senescence and the SASP in nearby non-senescent cells (Xu et al., Proc Natl Acad Sci USA 112:E6301-6310, 2015; Acosta et al., Nature Cell Biol 15:978-990, 2013; Nelson et al., Aging Cell 11:345-349, 2012; Xu et al., Nature Med 24:1246-1256, 2018; and da Silva et al., Aging Cell 18:e12848, 2019). Senescent cells also can directly impair the function of healthy stem cells (Xu et al., Elife 4:e12997, 2015). Paracrine signaling from senescent cells can amplify damage within tissues, possibly through the SASP, ROS, or other factors, which might partially explain why small number of senescent cells can be so harmful.

The INK-ATTAC (Baker et al., Nature 479:232-236, 2011) and p16-3MR (Demaria et al., Developmental Cell 31:722-733, 2014) transgenic mouse models have been used to investigate the role of senescent cells in vivo. Both models were designed using the p16 promoter to drive an inducible suicide gene, by which p16^{Ink4a}-highly-expressing (p16^{high} cells) can be eliminated in vivo. By leveraging these two models, the causal role of p16^{high} cells has been suggested in a number of pathological conditions, including osteoporosis (Farr et al., Nature Med 23:1072-1079, 2017), metabolic dysfunction (Xu et al., Elife 4:e12997, 2015; and Palmer et al., Aging Cell e12950, 2019), osteoarthritis (Jeon et al., Nature Med 23:775-781, 2017), neurodegenerative diseases (Bussian et al., Nature 562:578-582, 2018), cardiac dysfunction (Baker et al. 2016, supra), kidney dysfunction (Baker et al. 2016, supra), vasomotor dysfunction (Roos et al., Aging Cell 15:973-977, 2016), atherosclerosis (Childs et al., Science 354:472-477, 2016), liver steatosis (Ogrodnik et al., Nature Commun 8:15691, 2017), pulmonary fibrosis (Schafer et al., Nature Commun 8:14532, 2017), stem cell dysfunction (Xu et al., Elife 4:e12997, 2015; and Chang et al., Nature Med 22:78-83, 2016), and lifespan reduction (Baker et al. 2016, supra). Two additional senescence-related transgenic mouse models have knock-in Cre inserted into the native p16 locus (Grosse et al., Cell Metab 32:87-99 e86, 2020; and Omori et al., Cell Metab 32:814-828 e816, 2020). Although valuable, these models only target p16^{high} cells. However, not all p16^{high} cells are senescent (Hall et al., Aging 9:1867-1884, 2017; and Frescas et al., Cell Cycle 16:1526-1533, 2017), and not all senescent cells express high levels of p16.

### SUMMARY

Disclosed herein is a p21-Cre mouse model that contains a p21 promoter driving an inducible Cre polypeptide. The mouse model can enable the examination of p21^{Cip1}-highly-expressing (p21^{high}) cells, a previously unexplored senescent cell population. p21^{high} cells are distinct from p16^{high} cells in a number of aged tissues, and exhibit several characteristics typical of senescent cells. By crossing p21-Cre mice with different floxed mice, p21^{high} cells can be monitored, sorted, imaged, eliminated, or modulated in vivo. As demonstrated herein, for example, use of the p21-Cre mouse model showed that p21^{high} cells can be induced by various conditions, and that percentages of p21^{high} cells were higher (ranging from 1.5% to 10%) in a number of tissues in 23-month-old mice than the percentages (<1%) in 3-month-old mice. In addition, intermittent clearance of p21^{high} cells improved physical function in 23-month-old mice. Thus, the studies described herein demonstrated that the p21-Cre mouse model is a useful tool for studying p21^{high} cells to further understand the biology of senescent cells. Thus, this document provides methods and materials for modeling and studying cellular senescence.

For example, in some cases, this document provides transgenic non-human animals that can express a marker in senescent cells (e.g., p21^{high} senescent cells). In some cases, this document provides transgenic non-human animals that can be induced to express a marker at a level that is directly proportional to the amount of senescent cells (e.g., p21^{high} senescent cells) in the animals. In some cases, this document provides transgenic non-human animals that can be induced to delete senescent cells (e.g., p21^{high} senescent cells). As described herein, transgenic mice can be produced to contain nucleic acid that allows for the controlled expression, detection, and/or clearance of senescent cells (e.g., p21^{high} senescent cells) by, for example, controllably inducing apoptosis of senescent cells while inducing little, or no, apoptosis of non-senescent cells. For example, a transgenic non-human animal provided herein can be allowed to grow and develop for a period of time and then can be treated with a compound (e.g., tamoxifen) capable of inducing apoptosis of senescent cells within the transgenic animal while inducing little, or no, apoptosis of non-senescent cells within the transgenic animal. Clearance of senescent cells within a transgenic non-human animal can delay or reduce the likelihood of age-related disorders and can maximize healthy lifespan. In some cases, a transgenic non-human animal provided herein can include nucleic acid encoding a marker polypeptide (e.g., a fluorescent polypeptide such as a GFP) configured to be expressed by senescent cells with little, or no, expression by non-senescent cells.

In one aspect, this document features a transgenic non-human animal, the nucleated cells of which contain a transgene, where the transgene includes a p21 promoter sequence operably linked to a nucleic acid sequence encoding a Cre recombinase (Cre) polypeptide fused to a tamoxifen-inducible estrogen receptor (ER^{T2}) domain. The transgene can further contain, 3′ of the nucleic acid sequence encoding the Cre polypeptide fused to an ER^{T2} domain, an internal ribosome entry site (IRES) and a nucleotide sequence encoding a marker. The marker can be a green fluorescent protein (GFP). The non-human animal can be a mouse. The p21 promoter sequence can have at least 95% sequence identity with SEQ ID NO:1. The transgene can be within an H11 genomic locus of the non-human animal. Visceral fat cells, brain cells, intestine cells, heart cells, liver cells, and/or skeletal muscle cells of the animal can express the Cre polypeptide fused to an ER^{T2} domain. Visceral fat cells, brain cells, intestine cells, heart cells, liver cells, and/or skeletal muscle cells of the animal can express the Cre polypeptide fused to an ER^{T2} domain and said marker.

In another aspect, this document features a nucleic acid containing a p21 promoter sequence operably linked to a nucleotide sequence encoding a Cre polypeptide fused to an ER^{T2} domain. The nucleic acid can further contain, 3′ of the nucleotide sequence encoding a Cre polypeptide fused to an ER^{T2} domain, an IRES and a nucleotide sequence encoding a marker. The marker can be a GFP. The p21 promoter sequence can have at least 95% sequence identity with SEQ ID NO:1. The Cre polypeptide can have an amino acid sequence at least 95% identical to SEQ ID NO:4. The ER^{T2} domain can have an amino acid sequence at least 95% identical to SEQ ID NO:6.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 includes a series of uniform manifold approximation and projection (UMAP) plots showing expression levels of p21 (cdkn1a) and p16 (cdkn2a) in visceral fat, liver and heart in 18-30 months old mice. The figures were generated using the Tabula Muris Senis interactive platform (tabula-murissenis.ds.czbiohub.org/), and show that p16^{high} and p21^{high} cells are two distinct cell populations in aged tissues.

FIG. 2A is a schematic depicting a p21-Cre transgene-containing construct. FIG. 2B is a schematic showing genotyping primers. FIG. 2C includes a pair of images of gels showing PCR products for two sets of genotyping primers.

FIGS. 3A-3D relate to whole-body live imaging of p21^{high} cells in vivo using bioluminescence imaging. FIG. 3A is a schematic depicting the Hipp11 and ROSA26 loci of PL mice. FIG. 3B includes representative images showing LUC activity in doxorubicin (DOXO) or PBS treated PL mice (both male and female). r.l.u., relative luciferase units. FIG. 3C includes representative images showing LUC activity in regular chow diet- (RCD-) or high fat diet- (HFD-) fed PL male mice, and quantification of LUC activity. n=5 for both groups. FIG. 3D includes representative showing of LUC activity in young and old PL mice (both male and female), and a graph plotting LUC activity. n=14 for young and n=12 for old mice. Results are shown as means±s.e.m. * p<0.05; two-tailed, unpaired Student's t-test.

FIGS. 4A and 4B show that p21^{high} cells accumulate in various tissues with aging. FIG. 4A is a schematic depicting the Hipp11 and ROSA26 loci of PL mice PT mice. FIG. 4B includes representative micrographs of 6 tissues in young and old PT (male and female) mice. Red, tdTomato; blue, DAPI. The percentages of tdTomato+ cells are shown for each micrograph image (mean±s.e.m.).

FIGS. 5A-5D show that p21^{high} cells can be induced by aging, chemotherapy, and obesity. FIG. 5A includes flow cytometry plots and a graph showing the proportion of tdTomato+p21^{high} cells in the visceral fat and liver of 3-month-old (n=4) and 23-month-old (n=3) PT mice. FIG. 5B includes flow cytometry plots and a graph showing the proportion of GFP+ p21^{high} cells in 3-month-old (n=4) and 23-month-old (n=3) PT mice. Results plotted in the graphs are shown as means±s.e.m. * p<0.05; two-tailed, unpaired Student's t-test. FIG. 5C includes a pair of representative micrographs of visceral fat in 3-month-old PT mice treated with PBS or DOXO. Red, tdTomato; blue, DAPI. FIG. 5D includes a pair of representative micrographs of visceral fat in 5-month-old PT mice fed with RCD or HFD for 2 months. Red, tdTomato; blue, DAPI.

FIGS. 6A-6E show that p21^{high} cells exhibit features of cellular senescence. FIG. 6A is a graph plotting relative mRNA expression in GFP+ p21^{high} cells and GFP− cells sorted from stromal vascular fraction (SVF) from obese PL mice. FIG. 6B includes representative images and graphs indicating the cell size distribution and average cell size for GFP+ and GFP− cells. FIG. 6C includes representative images and graphs showing the mean SA-β-gal staining pixel distribution and the amount of SA-β-gal+ cells as a percentage of GFP+ and GFP− cells. FIG. 6D includes representative images and graphs showing mean EdU staining intensity distribution and the amount of EdU+ cells as a percentage of GFP+ and GFP− cells. FIG. 6E includes flow cytometry plots of Lamin B1 in GFP+ and GFP− cells. For FIG. 6A, n=7 for both groups. For FIGS. 6B-6E, n=5 for both groups. Results are shown as means±s.e.m. * p<0.05; two-tailed, paired Student's t-test.

FIGS. 7A-7C relate to inducible elimination of p21^{high} cells using diphtheria toxin A. FIG. 7A is a schematic depicting the Hipp11 and ROSA26 loci of PLD mice. FIG. 7B includes a representative image and a graph showing LUC activity in PL and PLD male mice fed with a HFD. n=5 for all groups. FIG. 7C includes a representative images and a graph showing quantification of LUC activity in 23-month-old PL and PLD mice (both male and female). n=14 for Y-PL, n=12 for O-PL, and n=11 for O-PLD mice. Results are shown as means±s.e.m. * p<0.05; two-tailed, unpaired Student's t-test. (Y: young, O: old)

FIGS. 8A-8D relate to genetic inhibition of SASP in p21^{high} cells in vivo. FIG. 8A is a schematic of wild type and mutant Rela alleles in floxed mice. FIG. 8B is a schematic depicting Rela loci in P-Rela and Rela mice. FIG. 8C includes representative images showing PCR results using Rela-F, Rela-WT-R, and Rela-Cut-R primers for SVF from P-Rela and Rela mice. FIG. 8D is a graph plotting relative mRNA expression in senescent WT and CAG-Rela ear fibroblasts. n=6 for both groups. Results are shown as means±s.e.m. * p<0.05; two-tailed, unpaired Student's t-test.

FIGS. 9A-9G show that clearance of p21^{high} cells improves physical function in old mice. FIG. 9A is a schematic illustrating the experimental design. FIG. 9B includes a pair of graphs plotting body weight and body weight change in PL and PLD mice (both male and female) at age 20 months (before tamoxifen treatment) and 23 months (after tamoxifen treatment). FIG. 9C includes a pair of graphs plotting maximal walking speed and walking speed as a percentage of baseline in PL and PLD mice (both male and female) at age 20 months (before tamoxifen treatment) and 23 months (after tamoxifen treatment). FIG. 9D includes a pair of graphs plotting grip strength and grip strength as a percentage of baseline in PL and PLD mice (both male and female) at age 20 months (before tamoxifen treatment) and 23 months (after tamoxifen treatment). FIG. 9E is a graph plotting hanging endurance at age 23 months. FIG. 9F is a graph plotting food intake FIG. 9G is a graph plotting daily activity at age 23 months. n=9 for both groups. Results are shown as means±s.e.m. * p<0.05; two-tailed, unpaired Student's t-test.

### DETAILED DESCRIPTION

This document relates to methods and materials involved in the evaluation and/or removal of senescent cells within a mammal. For example, this document provides transgenic non-human animals that can contain an exogenous nucleic acid that includes a p21 promoter sequence operably linked to a sequence encoding an inducible Cre polypeptide. Such non-human animals can be farm animals such as pigs, goats, sheep, cows, horses, and rabbits, rodents such as rats, guinea pigs, and mice, and non-human primates such as baboons, monkeys, and chimpanzees. The term “transgenic non-human animal” as used herein includes, without limitation, founder transgenic non-human animals as well as progeny of the founders, progeny of the progeny, and so forth, provided that the progeny retain the transgene. The nucleated cells of the transgenic non-human animals provided herein can contain a transgene that includes a p21 promoter sequence operably linked to a nucleic acid sequence encoding an inducible Cre polypeptide, where the Cre polypeptide is capable of causing recombination at loxP sites flanking a nucleotide sequence of interest at another genomic site within the animal. A P21 promoter sequence of a transgene described herein can drive Cre polypeptide expression in senescent cells while driving less, little, or no Cre polypeptide expression in non-senescent cells.

In some cases, the inducible Cre polypeptide capable of causing recombination at loxP sites can be a polypeptide that includes two polypeptide sequences fused together (e.g., a fusion polypeptide). For example, a fusion polypeptide can include a Cre polypeptide and an inducible estrogen receptor (ER) polypeptide (e.g., a tamoxifen-inducible ER polypeptide (ER^{T2}), as described herein). In some cases, a transgene provided herein can further include nucleic acid encoding a marker polypeptide such as a fluorescent polypeptide (e.g., GFP, BFP, or RFP). For example, a transgene provided herein can include a p21 promoter operably linked to a sequence encoding a fusion polypeptide that includes Cre and an inducible ER polypeptide, followed by an internal ribosome entry site (IRES), followed by a sequence encoding a marker polypeptide (e.g., GFP).

The term “operably linked” as used herein refers to positioning a regulatory element (e.g., a promoter sequence, an inducible element, or an enhancer sequence) relative to a nucleic acid sequence encoding a polypeptide in such a way as to permit or facilitate expression of the encoded polypeptide. In the transgenes disclosed herein, for example, a promoter sequence (e.g., a p21 promoter sequence) can be positioned 5′ relative to a nucleic acid encoding a polypeptide (e.g., a Cre-ER^{T2} fusion polypeptide).

This document provides transgene nucleic acids, as well as transgenic non-human animals containing the transgenes, and methods for using the transgenic non-human animals. The transgene nucleic acids provided herein can contain a p21 promoter operably linked to a sequence encoding an inducible Cre polypeptide. In some cases, a transgene nucleic acid provided herein also can include an IRES and a nucleotide sequence encoding a marker.

The p21 promoter in the transgene constructs and transgenic non-human animals provided herein can have any appropriate length and any appropriate sequence, provided that it drives expression of an operably linked coding sequence under conditions that induce senescence. For example, a p21 promoter can be from about 3000 to about 3500 nucleotides in length (e.g., about 3000 to about 3100, about 3100 to about 3200, about 3200 to about 3300, about 3300 to about 3400, or about 3400 to about 3500 nucleotides in length). Further, the p21 promoter can have the sequence set forth in SEQ ID NO:1, or can have a sequence that is at least 95% identical (e.g., at least 96%, at least 97%, at least 98%, or at least 99% identical) to the p21 promoter sequence set forth in SEQ ID NO:1:

(SEQ ID NO: 1)
ctatcctgaccctcgtgcttagaccattttctattctctctttgttttgt
ggtcgaatttcttgggagtttgtgtggaggtgacttcttctgaaatctga
cagtccctctttggtactcccctgtccttttctgggaagtggtgattttg
gcgtccacacttcctcttccttcctgggtagcagcaacagctaacatgga
gtcttccttatatctcccttggtcccttggatttcctttctatcagcccc
agaggataccttgcaaggctgcatcagtcctcccatccctggctgttgcc
tctcggagaccagcagcaaaatcggagctcagcaggcctgggtctgttca
gtcctgggtggggactagctttctggccttcaggaacatgtcttgacatg
ttcagccctggaattgaagaggtggggctgcttcagtgcagggtggtgga
gacctgatgatacccaactaccagctgtggggtgaggaggagcatgaatg
gagacagagaccccagataattaaggacgtcccactttgccagcagaata
aaaggtggtatgtatcttgtgacatgtatcaggtgaaggatgcttttgtg
catctgtgtgtgtgtgcctggggtgtgtatgtgtcaggtactgtatgtag
tcattttgtcactttgtcattttggggtctggagggctcctccaaccatg
tttctgagtatacattcacgtgcaatggtgtgcctgactatacattcaag
tgcaaggccaagaatgtttgttagaaagactgagtagtcccagacttaat
aaatatttgttgagtacttttgtggtgctctgggaagccagaagttgttt
aaaataaatctctccaacaccagtagggtaaaggcacaggaggtcacagc
actcagcagttcagtataagcctttattcaagctgttttctcccaaagta
aacagacagacaatgtcacttctatctgagaagcctggaggccaagggga
tttgggcagttttgacatcctgtgctggcccctgacagcccagccctggg
atggacgacttggatgcagggactggaccgttcaggagctggggcattgt
gggagtggccattatgtctgtcctggtttgggggtctgaagggggtcctt
caactgtgtttctgaacaggatgaggcttttgaggggggttgggaaggtg
gccaagcccttcccagacttccaccccccatcacagaagaggaggcctgt
ctaggtcagctaaatccgaggaggaagactgggcatgtctgggcagcgat
ctctagacatcggagagcagatgtcagaactcacagcttctccaaagcag
gattttgatcttttaactaaagatatccgttcaaactaagactccagtct
ctgctttatttaaaatttttgtttgtgtttgttttgagagagagaggaat
ttgtttttgttttagaggcaggatgtcatgtaaccttgatgaattattgg
tcctcctgtttctgtctcctgagtgccgggattacagatgtatgccgcta
tcatctagatgatgccttactagggatccaactcctggcttcatacatgt
taggcaagcgctatattaacggagctacatccctttttggatgcatggtg
atctcagatagctcaggctagccttgagctccaaatcccccctgcctccc
aagtaccgtgatttcaggcatgtacctctatgcttagctgagatggtggt
cttgctatgtagcccatgtgaccaggctggatcgtgtaacaagactgaag
aaaacccccttctgctgggtgtgatggctcagacctgtagtcttaactct
aagcaaggaagttaaggcaggaagattgccttgaatatgagggccaccct
gggctacatagcgaggcctcgtctcaaaagaccccaaacagaaggaaata
aaactgactagagacatggaggaaggtgggaacggagaatgtcttactgc
tatgtctgtcaggaacatccgtagatgtttctagaattgtcctttatcaa
tgtcattttagtggactgtctggatcttgggagggggagtattagacatt
tccctcattttggacccagagaaagaaatctgcaagcagagtactctggg
cagcttgccagaggtcagcaggtagccattagtgtggtcccagtcaggtc
ttgatgctctcacttgcaggatgtattatggtgtgagaaatgttcacatg
ctggcttctgaagaggggagaggggaggtaaggagcctggcggctgtttt
tcttggtagtccgtggtctgagaactggactcaatctccccgatttctga
ggcggttgacagcatcctttccttctgtggaactgctttcctcgtctgtg
agacagggaggaaatgatcgcgttctggacccgatgtccgaggggcttct
gggaggagggggaaaaaaatctccagacatagtgggacttcttgggattt
taaactattttttattatttatgggcttgttttgtttttgagagggtctc
aatggatagcccaggctggtcttgaacctgtaacgcccctcgtgcctcaa
tctcccaagtataggattccaggcttttgctatcatactcaaatgatcaa
tttatttatatttgaaacagtgtcacatatttcaagttggtctccatcgg
aataggtagctgtcaaaacgaccttgaatgcctatttccccctcctcacc
ccccactgggcgctgttattacagacgtgaccccgcatgcccagtttatg
gggccctggagctccaacccagggcttcacttccagcaagttaggcaaac
actgtaccaacagacccacctcccgaaacccaggattttatttactaata
tcagtgatctggaaaagagttagtccttcccacagttggtcagggacaga
cccataaacactcactcagctctaactgtactgttgttcatagatgtatg
tggctctgctggtgcgctgcgtgacaagagaatagcccaggtgtggggga
ggggagggcgcgccctcttaacgcgcgccggttctagctgtctggcgcgg
gcttagattcccagaggggagggcgggccagcgagtccccgggatcggtg
aaggagtgggttggtcctgcctctgagggggcggggcctgggccgacgct
ataaggaggcagctcgacgccaac.

The sequence encoding an inducible Cre polypeptide within the transgene constructs and transgenic non-human animals provided herein can have any appropriate length and any appropriate sequence, provided that after expression and induction, the Cre polypeptide can cause recombination at loxP sites within the cells in which the Cre polypeptide is expressed. In some cases, for example, an inducible Cre polypeptide can be a Cre-ER^{T2} fusion polypeptide. The Cre-ER^{T2} fusion polypeptide can have the amino acid sequence set forth in SEQ ID NO:2, or can have a sequence that is at least 95% identical (e.g., at least 96%, at least 97%, at least 98%, or at least 99% identical) to the amino acid sequence set forth in SEQ ID NO:2:

(SEQ ID NO: 2)
MSNLLTVHQNLPALPVDATSDEVRKNLMDMFRDRQAFSEHTWKMLLSVCR
SWAAWCKLNNRKWFPAEPEDVRDYLLYLQARGLAVKTIQQHLGQLNMLHR
RSGLPRPSDSNAVSLVMRRIRKENVDAGERAKQALAFERTDFDQVRSLME
NSDRCQDIRNLAFLGIAYNTLLRIAEIARIRVKDISRTDGGRMLIHIGRT
KTLVSTAGVEKALSLGVTKLVERWISVSGVADDPNNYLFCRVRKNGVAAP
SATSQLSTRALEGIFEATHRLIYGAKDDSGQRYLAWSGHSARVGAARDMA
RAGVSIPEIMQAGGWTNVNIVMNYIRNLDSETGAMVRLLEDGDLEPSAGD
MRAANLWPSPLMIKRSKKNSLALSLTADQMVSALLDAEPPILYSEYDPTR
PFSEASMMGLLTNLADRELVHMINWAKRVPGFVDLTLHDQVHLLECAWLE
ILMIGLVWRSMEHPVKLLFAPNLLLDRNQGKCVEGMVEIFDMLLATSSRF
RMMNLQGEEFVCLKSIILLNSGVYTFLSSTLKSLEEKDHIHRVLDKITDT
LIHLMAKAGLTLQQQHQRLAQLLLILSHIRHMSNKGMEHLYSMKCKNVVP
LYDLLLEAADAHRLHAPTSRGGASVEETDQSHLATAGSTSSHSLQKYYIT
GEAEGFPATA

In some cases, the Cre-ER^{T2} polypeptide can be encoded by the nucleotide sequence set forth in SEQ ID NO:3, or can be encoded by a nucleotide sequence that is at least 95% identical (e.g., at least 96%, at least 97%, at least 98%, or at least 99% identical) to the Cre-ER^{T2} nucleotide sequence set forth in SEQ ID NO:3:

(SEQ ID NO: 3)
atgtccaatttactgaccgtacaccaaaatttgcctgcattaccggtcga
tgcaacgagtgatgaggttcgcaagaacctgatggacatgttcagggatc
gccaggcgttttctgagcatacctggaaaatgcttctgtccgtttgccgg
tcgtgggcggcatggtgcaagttgaataaccggaaatggtttcccgcaga
acctgaagatgttcgcgattatcttctatatcttcaggcgcgcggtctgg
cagtaaaaactatccagcaacatttgggccagctaaacatgcttcatcgt
cggtccgggctgccacgaccaagtgacagcaatgctgtttcactggttat
gcggcggatccgaaaagaaaacgttgatgccggtgaacgtgcaaaacagg
ctctagcgttcgaacgcactgatttcgaccaggttcgttcactcatggaa
aatagcgatcgctgccaggatatacgtaatctggcatttctggggattgc
ttataacaccctgttacgtatagccgaaattgccaggatcagggttaaag
atatctcacgtactgacggtgggagaatgttaatccatattggcagaacg
aaaacgctggttagcaccgcaggtgtagagaaggcacttagcctgggggt
aactaaactggtcgagcgatggatttccgtctctggtgtagctgatgatc
cgaataactacctgttttgccgggtcagaaaaaatggtgttgccgcgcca
tctgccaccagccagctatcaactcgcgccctggaagggatttttgaagc
aactcatcgattgatttacggcgctaaggatgactctggtcagagatacc
tggcctggtctggacacagtgcccgtgtcggagccgcgcgagatatggcc
cgcgctggagtttcaataccggagatcatgcaagctggtggctggaccaa
tgtaaatattgtcatgaactatatccgtaacctggatagtgaaacagggg
caatggtgcgcctgctggaagatggcgatctcgagccatctgctggagac
atgagagctgccaacctttggccaagcccgctcatgatcaaacgctctaa
gaagaacagcctggccttgtccctgacggccgaccagatggtcagtgcct
tgttggatgctgagccccccatactctattccgagtatgatcctaccaga
cccttcagtgaagcttcgatgatgggcttactgaccaacctggcagacag
ggagctggttcacatgatcaactgggcgaagagggtgccaggctttgtgg
atttgaccctccatgatcaggtccaccttctagaatgtgcctggctagag
atcctgatgattggtctcgtctggcgctccatggagcacccagtgaagct
actgtttgctcctaacttgctcttggacaggaaccagggaaaatgtgtag
agggcatggtggagatcttcgacatgctgctggctacatcatctcggttc
cgcatgatgaatctgcagggagaggagtttgtgtgcctcaaatctattat
tttgcttaattctggagtgtacacatttctgtccagcaccctgaagtctc
tggaagagaaggaccatatccaccgagtcctggacaagatcacagacact
ttgatccacctgatggccaaggcaggcctgaccctgcagcagcagcacca
gcggctggcccagctcctcctcatcctctcccacatcaggcacatgagta
acaaaggcatggagcatctgtacagcatgaagtgcaagaacgtggtgccc
ctctatgacctgctgctggaggcggcggacgcccaccgcctacatgcgcc
cactagccgtggaggggcatccgtggaggagacggaccaaagccacttgg
ccactgcgggctctacttcatcgcattccttgcaaaagtattacatcacg
ggggaggcagagggtttccctgccacagcttgataacgcgtttaattaac
tcgaggttttcgaggtcgacggtatcgataagcttgatatcgaattccg

In some cases, the Cre portion of the Cre-ER^{T2} fusion polypeptide can have the amino acid sequence set forth in SEQ ID NO:4, or can have a sequence that is at least 95% identical (e.g., at least 96%, at least 97%, at least 98%, or at least 99% identical) to the amino acid sequence set forth in SEQ ID NO:4:

(SEQ ID NO: 4)
MSNLLTVHQNLPALPVDATSDEVRKNLMDMFRDRQAFSEHTWKMLLSVCR
SWAAWCKLNNRKWFPAEPEDVRDYLLYLQARGLAVKTIQQHLGQLNMLHR
RSGLPRPSDSNAVSLVMRRIRKENVDAGERAKQALAFERTDFDQVRSLME
NSDRCQDIRNLAFLGIAYNTLLRIAEIARIRVKDISRTDGGRMLIHIGRT
KTLVSTAGVEKALSLGVTKLVERWISVSGVADDPNNYLFCRVRKNGVAAP
SATSQLSTRALEGIFEATHRLIYGAKDDSGQRYLAWSGHSARVGAARDMA
RAGVSIPEIMQAGGWTNVNIVMNYIRNLDSETGAMVRLLEDGD

The Cre portion of the Cre-ER^{T2} polypeptide can be encoded by the nucleotide sequence set forth in SEQ ID NO:5, or can be encoded by a nucleotide sequence that is at least 95% identical (e.g., at least 96%, at least 97%, at least 98%, or at least 99% identical) to the nucleotide sequence set forth in SEQ ID NO:5:

(SEQ ID NO: 5)
atgtccaatttacttaccgtacaccaaaatttgcctgcattaccggtcga
tgcaacgagtgatgaggttcgcaagaacctgatggacatgttcagggatc
gccaggcgttttctgagcatacctggaaaatgcttctgtccgtttgccgg
tcgtgggcggcatggtgcaagttgaataaccggaaatggtttcccgcaga
acctgaagatgttcgcgattatcttctatatcttcaggcgcgcggtctgg
cagtaaaaactatccagcaacatttgggccagctaaacatgcttcatcgt
cggtccgggctgccacgaccaagtgacagcaatgctgtttcactggttat
gcggcggatccgaaaagaaaacgttgatgccggtgaacgtgcaaaacagg
ctctagcgttcgaacgcactgatttcgaccaggttcgttcactcatggaa
aatagcgatcgctgccaggatatacgtaatctggcatttctggggattgc
ttataacaccctgttacgtatagccgaaattgccaggatcagggttaaag
atatctcacgtactgacggtgggagaatgttaatccatattggcagaacg
aaaacgctggttagcaccgcaggtgtagagaaggcacttagcctgggggt
aactaaactggtcgagcgatggatttccgtctctggtgtagctgatgatc
cgaataactacctgttttgccgggtcagaaaaaatggtgttgccgcgcca
tctgccaccagccagctatcaactcgcgccctggaagggatttttgaagc
aactcatcgattgatttacggcgctaaggatgactctggtcagagatacc
tggcctggtctggacacagtgcccgtgtcggagccgcgcgagatatggcc
cgcgctggagtttcaataccggagatcatgcaagctggtggctggaccaa
tgtaaatattgtcatgaactatatccgtaacctggatagtgaaacagggg
caatggtgcgcctgctggaagatggcgattag

In some cases, the ER^{T2} portion of the Cre-ER^{T2} fusion polypeptide can have the amino acid sequence set forth in SEQ ID NO:6, or can have a sequence that is at least 95% identical (e.g., at least 96%, at least 97%, at least 98%, or at least 99% identical) to the amino acid sequence set forth in SEQ ID NO:6:

(SEQ ID NO: 6)
MRAANLWPSPLMIKRSKKNSLALSLTADQMVSALLDAEPPILYSEYDPTR
PFSEASMMGLLTNLADRELVHMINWAKRVPGFVDLTLHDQVHLLECAWLE
ILMIGLVWRSMEHPVKLLFAPNLLLDRNQGKCVEGMVEIFDMLLATSSRF
RMMNLQGEEFVCLKSIILLNSGVYTFLSSTLKSLEEKDHIHRVLDKITDT
LIHLMAKAGLTLQQQHQRLAQLLLILSHIRHMSNKGMEHLYSMKCKNVVP
LYDLLLEAADAHRLHAPTSRGGASVEETDQSHLATAGSTSSHSLQKYYIT
GEAEGFPATA

In some cases, the ER^{T2} portion of the Cre-ER^{T2} polypeptide can be encoded by the nucleotide sequence set forth in SEQ ID NO:7, or can be encoded by a nucleotide sequence that is at least 95% identical (e.g., at least 96%, at least 97%, at least 98%, or at least 99% identical) to the ER^{T2} nucleotide sequence set forth in SEQ ID NO:7:

(SEQ ID NO: 7)
ctcgagccatctgctggagacatgagagctgccaacctttggccaagccc
gctcatgatcaaacgctctaagaagaacagcctggccttgtccctgacgg
ccgaccagatggtcagtgccttgttggatgctgagccccccatactctat
tccgagtatgatcctaccagacccttcagtgaagcttcgatgatgggctt
actgaccaacctggcagacagggagctggttcacatgatcaactgggcga
agagggtgccaggctttgtggatttgaccctccatgatcaggtccacctt
ctagaatgtgcctggctagagatcctgatgattggtctcgtctggcgctc
catggagcacccagtgaagctactgtttgctcctaacttgctcttggaca
ggaaccagggaaaatgtgtagagggcatggtggagatcttcgacatgctg
ctggctacatcatctcggttccgcatgatgaatctgcagggagaggagtt
tgtgtgcctcaaatctattattttgcttaattctggagtgtacacatttc
tgtccagcaccctgaagtctctggaagagaaggaccatatccaccgagtc
ctggacaagatcacagacactttgatccacctgatggccaaggcaggcct
gaccctgcagcagcagcaccagcggctggcccagctcctcctcatcctct
cccacatcaggcacatgagtaacaaaggcatggagcatctgtacagcatg
aagtgcaagaacgtggtgcccctctatgacctgctgctggaggcggcgga
cgcccaccgcctacatgcgcccactagccgtggaggggcatccgtggagg
agacggaccaaagccacttggccactgcgggctctacttcatcgcattcc
ttgcaaaagtattacatcacgggggaggcagagggtttccctgccacagc
ttgataacgcgtttaattaactcgaggttttcgaggtcgacggtatcgat
aagcttgatatcgaattccg

When an IRES and a nucleotide sequence encoding a marker are included in the transgene constructs and transgenic non-human animals provided herein, any appropriate IRES sequence and marker coding sequence can be used. For example, a IRES can have the sequence set forth in SEQ ID NO:8, or a sequence that is at least 95% identical (e.g., at least 96%, at least 97%, at least 98%, or at least 99% identical) to the IRES sequence set forth in SEQ ID NO: 8:

(SEQ ID NO: 8)
cccctctccctcccccccccctaacgttactggccgaagccgcttggaat
aaggccggtgtgcgtttgtctatatgttattttccaccatattgccgtct
tttggcaatgtgagggcccggaaacctggccctgtcttcttgacgagcat
tcctaggggtctttcccctctcgccaaaggaatgcaaggtctgttgaatg
tcgtgaaggaagcagttcctctggaagcttcttgaagacaaacaacgtct
gtagcgaccctttgcaggcagcggaaccccccacctggcgacaggtgcct
ctgcggccaaaagccacgtgtataagatacacctgcaaaggcggcacaac
cccagtgccacgttgtgagttggatagttgtggaaagagtcaaatggctc
tcctcaagcgtattcaacaaggggctgaaggatgcccagaaggtacccca
ttgtatgggatctgatctggggcctcggtgcacatgctttacatgtgttt
agtcgaggttaaaaaaacgtctaggccccccgaaccacggggacgtggtt
ttcctttgaaaaacacgatgataa

In some cases, the marker can be a fluorescent marker. For example, the marker can be a GFP, and can have the amino acid sequence set forth in SEQ ID NO:9, or can have an amino sequence that is at least 95% identical (e.g., at least 96%, at least 97%, at least 98%, or at least 99% identical) to the sequence set forth in SEQ ID NO:9:

(SEQ ID NO: 9)
MVSKGEELFTGVVPILVELDGDVNGHKFSVSGEGEGDATYGKLTLKFICT
TGKLPVPWPTLVTTLTYGVQCFSRYPDHMKQHDFFKSAMPEGYVQERTIF
FKDDGNYKTRAEVKFEGDTLVNRIELKGIDFKEDGNILGHKLEYNYNSHN
VYIMADKQKNGIKVNFKIRHNIEDGSVQLADHYQQNTPIGDGPVLLPDNH
YLSTQSALSKDPNEKRDHMVLLEFVTAAGITLGMDELYK

In some cases, the GFP can be encoded by the nucleotide sequence set forth in SEQ ID NO: 10, or by a nucleotide sequence that is at least 95% identical (e.g., at least 96%, at least 97%, at least 98%, or at least 99% identical) to the sequence set forth in SEQ ID NO: 10:

(SEQ ID NO: 10)
tatggccacaaccatggtgagcaagggcgaggagctgttcaccggggtgg
tgcccatcctggtcgagctggacggcgacgtaaacggccacaagttcagc
gtgtccggcgagggcgagggcgatgccacctacggcaagctgaccctgaa
gttcatctgcaccaccggcaagctgcccgtgccctggcccaccctcgtga
ccaccctgacctacggcgtgcagtgcttcagccgctaccccgaccacatg
aagcagcacgacttcttcaagtccgccatgcccgaaggctacgtccagga
gcgcaccatcttcttcaaggacgacggcaactacaagacccgcgccgagg
tgaagttcgagggcgacaccctggtgaaccgcatcgagctgaagggcatc
gacttcaaggaggacggcaacatcctggggcacaagctggagtacaacta
caacagccacaacgtctatatcatggccgacaagcagaagaacggcatca
aggtgaacttcaagatccgccacaacatcgaggacggcagcgtgcagctc
gccgaccactaccagcagaacacccccatcggcgacggccccgtgctgct
gcccgacaaccactacctgagcacccagtccgccctgagcaaagacccc

The percent sequence identity between a particular nucleic acid or amino acid sequence and a sequence referenced by a particular sequence identification number is determined as follows. First, a nucleic acid or amino acid sequence is compared to the sequence set forth in a particular sequence identification number using the BLAST 2 Sequences (Bl2seq) program from the stand-alone version of BLASTZ containing BLASTN version 2.0.14 and BLASTP version 2.0.14. This stand-alone version of BLASTZ can be obtained online at fr.com/blast or at ncbi.nlm.nih.gov. Instructions explaining how to use the Bl2seq program can be found in the readme file accompanying BLASTZ. Bl2seq performs a comparison between two sequences using either the BLASTN or BLASTP algorithm. BLASTN is used to compare nucleic acid sequences, while BLASTP is used to compare amino acid sequences. To compare two nucleic acid sequences, the options are set as follows: -i is set to a file containing the first nucleic acid sequence to be compared (e.g., C:\seq1.txt); -j is set to a file containing the second nucleic acid sequence to be compared (e.g., C:\seq2.txt); -p is set to blastn; -o is set to any desired file name (e.g., C:\output.txt); -q is set to -1; -r is set to 2; and all other options are left at their default setting. For example, the following command can be used to generate an output file containing a comparison between two sequences: C:\Bl2seq -i c:\seq1.txt -j c:\seq2.txt -p blastn -o c:\output.txt -q -1 -r 2. To compare two amino acid sequences, the options of Bl2seq are set as follows: -i is set to a file containing the first amino acid sequence to be compared (e.g., C:\seq1.txt); -j is set to a file containing the second amino acid sequence to be compared (e.g., C:\seq2.txt); -p is set to blastp; -o is set to any desired file name (e.g., C:\output.txt); and all other options are left at their default setting. For example, the following command can be used to generate an output file containing a comparison between two amino acid sequences: C:\Bl2seq -i c:\seq1.txt -j c:\seq2.txt -p blastp -o c:\output.txt. If the two compared sequences share homology, then the designated output file will present those regions of homology as aligned sequences. If the two compared sequences do not share homology, then the designated output file will not present aligned sequences.

Once aligned, the number of matches is determined by counting the number of positions where an identical nucleotide or amino acid residue is presented in both sequences. The percent sequence identity is determined by dividing the number of matches either by the length of the sequence set forth in the identified sequence (e.g., SEQ ID NO:1), or by an articulated length (e.g., 20 consecutive nucleotides or amino acid residues from a sequence set forth in an identified sequence), followed by multiplying the resulting value by 100. For example, an amino acid sequence that has 3100 matches when aligned with the sequence set forth in SEQ ID NO:1 is 96.2 percent identical to the sequence set forth in SEQ ID NO:1 (i.e., 3100÷3224×100=96.2). It is noted that the percent sequence identity value is rounded to the nearest tenth. For example, 75.11, 75.12, 75.13, and 75.14 is rounded down to 75.1, while 75.15, 75.16, 75.17, 75.18, and 75.19 is rounded up to 75.2. It also is noted that the length value will always be an integer.

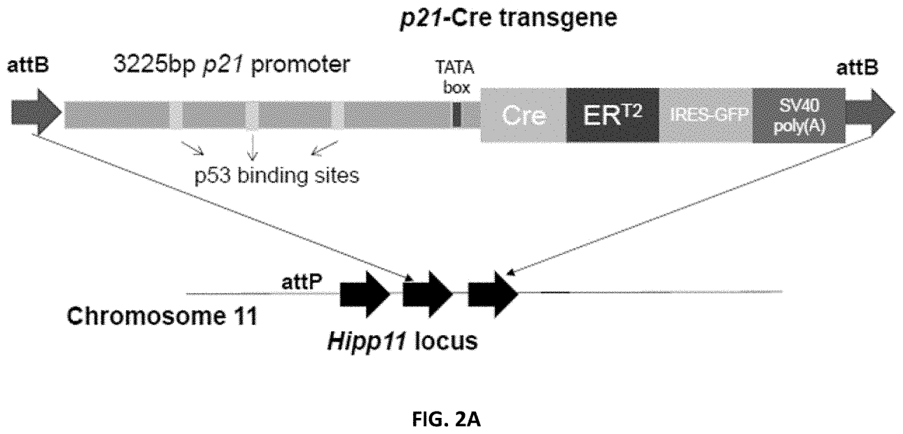

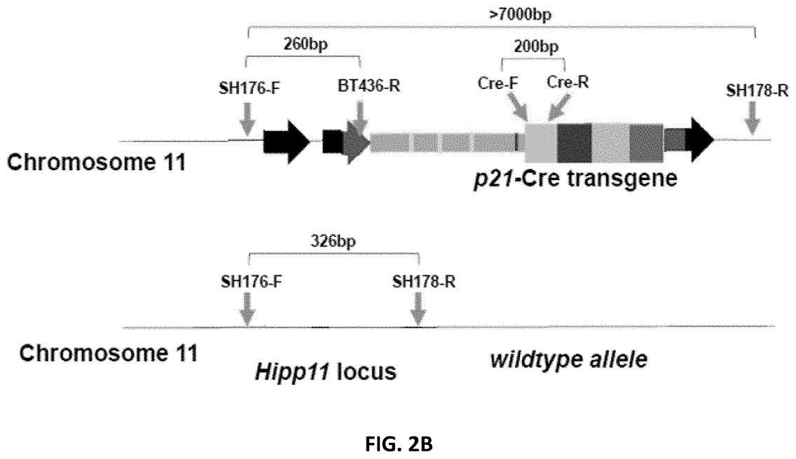

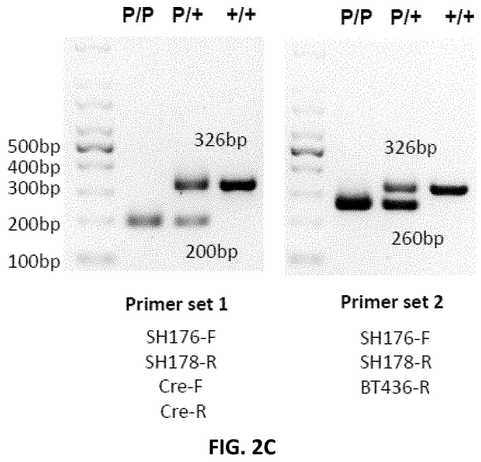

Various techniques known in the art can be used to introduce transgenes into non-human animals to produce founder lines, in which the transgene is integrated into the genome. Such techniques include, without limitation, pronuclear microinjection (see, e.g., U.S. Pat. No. 4,873,191), retrovirus mediated gene transfer into germ lines (Van der Putten et al., Proc. Natl. Acad. Sci. USA 82:6148-1652, 1985), gene targeting into embryonic stem cells (Thompson et al., Cell 56:313-321, 1989), electroporation of embryos (Lo, Mol. Cell. Biol. 3:1803-1814, 1983), and in vitro transformation of somatic cells, such as cumulus or mammary cells, followed by nuclear transplantation (Wilmut et al., Nature 385:810-813, 1997; and Wakayama et al., Nature 394:369-374, 1998). For example, fetal fibroblasts can be genetically modified to contain a p21-Cre construct (FIG. 2A), and then fused with enucleated oocytes. After activation of the oocytes, the eggs can be cultured to the blastocyst stage. See, for example, Cibelli et al., Science 280:1256-1258, 1998). Standard breeding techniques can be used to create animals that are homozygous for the transgene from the initial heterozygous founder animals. Homozygosity is not necessarily required, however, as the phenotype can be observed in hemizygotic animals.

Once transgenic non-human animals have been generated, expression of an encoded polypeptide (e.g., a Cre-ER^{T2} fusion protein and/or a marker polypeptide) can be assessed using any appropriate technique. For example, initial screening can be accomplished by Southern blot analysis to determine whether or not integration of the transgene has taken place. For a description of Southern analysis, see sections 9.37-9.52 of Sambrook et al., 1989, Molecular Cloning, A Laboratory Manual, second edition, Cold Spring Harbor Press, Plainview; NY. Polymerase chain reaction (PCR) techniques also can be used for initial screening. PCR is described in, for example PCR Primer: A Laboratory Manual, ed. Dieffenbach and Dveksler, Cold Spring Harbor Laboratory Press, 1995. Nucleic acids also can be amplified by ligase chain reaction, strand displacement amplification, self-sustained sequence replication, or nucleic acid sequence-based amplified. See, for example, Lewis, Genetic Engineering News 12:1, 1992; Guatelli et al., Proc. Natl. Acad. Sci. USA 87:1874-1878, 1990; and Weiss, Science 254:1292-1293, 1991).

Expression of a nucleic acid sequence encoding a polypeptide (e.g., an inducible Cre polypeptide such as a Cre-ER^{T2} fusion polypeptide, or a marker polypeptide) in cells of transgenic non-human animals can be assessed using techniques that include, without limitation, Northern blot analysis of tissue samples obtained from the animal, in situ hybridization analysis, Western blot analysis, immunoassays such as enzyme-linked immunosorbent assays, and reverse-transcriptase PCR (RT-PCR).

In some cases, as described herein, transgenic non-human animals containing a p21-Cre transgene can be crossed with other transgenic non-human animals containing a transgene that encodes a reporter (e.g., luciferase or tdTomato) or another polypeptide (e.g., a diphtheria toxin polypeptide, such as a diphtheria toxin A polypeptide), where the nucleotide sequence encoding the reporter or other polypeptide is separated from a promoter by a loxP-flanked STOP fragment. Progeny of the cross that are doubly transgenic can be selected and evaluated as described in the Examples herein. For example, expression and inducement of an inducible Cre polypeptide (e.g., a Cre-ER^{T2} fusion polypeptide) by cells within a doubly transgenic animal can lead to removal of the floxed STOP fragment and expression of the reporter or other polypeptide. It is understood that a particular phenotype in a transgenic or doubly transgenic animal typically is assessed by comparing the phenotype in the transgenic animal to the corresponding phenotype exhibited by a control non-human animal that lacks the transgene.

A transgenic non-human animal provided herein can have any appropriate genetic background.

This document also provides tissues (e.g., adipose, liver, intestine, brain, heart, and muscle) and cells (e.g., fat cells, preadipocytes, muscle cells, neuronal progenitor cells, hepatocytes, and endothelial cells) obtained from a transgenic non-human animal provided herein. Any appropriate method can be used to obtain senescent cells from a mammal. For example, senescent cells expressing a marker polypeptide (e.g., GFP) under the control of a p21 promoter sequence can be separated from non-senescent cells using techniques such as cell sorting methods based on the expression of the marker polypeptide. In some cases, cell lines of senescent cells can be used in place of freshly obtained senescent cells to identify agents having the ability to kill, or to facilitate the killing of, senescent cells and agents having the ability to delay, or reduce the likelihood of age-related disorders, and/or maximize healthy lifespan as described herein. In some cases, senescent cells can be obtained by cell passage in culture (e.g., greater than about 12, greater than about 15, or greater than about 20 cell passages). In some cases, senescent cells can be obtained from p21-Cre transgenic non-human animals fed a HFD or treated with DOXO. In some cases, senescent cells can be obtained from older p21-Cre transgenic non-human animals (e.g., p21-Cre mice that are at least 18, at least 20, or at least 23 months of age).

In some cases, senescent cells can be exposed to one or more test agents to identify agents having the ability to kill, or to facilitate the killing of, the senescent cells. Once identified as having the ability to kill, or to facilitate the killing of, the senescent cells, the identified agent can be applied to comparable non-senescent cells in comparable concentrations to confirm that the agent has a reduced ability to kill, or to facilitate the killing of, non-senescent cells. Those agents having the ability to kill, or to facilitate the killing of, senescent cells, with a reduced or no ability to kill, or to facilitate the killing of, non-senescent cells, can be classified as being agents having the ability to delay, or reduce the likelihood of age-related disorders, and/or maximize healthy lifespan. In some cases, senescent cells obtained from a transgenic mammal provided herein and treated in a manner that results in senescent cell death can be used as positive controls.

This document also provides methods and materials for identifying molecules (e.g., polypeptides, carbohydrates, lipids, and nucleic acids) possessed or expressed by senescent cells. For example, senescent cells can be obtained as described herein and assessed to identify molecules (e.g., polypeptides) possessed or expressed by those senescent cells. Any appropriate method can be used to identify molecules possessed or expressed by senescent cells. In some cases, for example, RNA-seq analysis can be used to identify molecules expressed by senescent cells. In some cases, polypeptide isolation and sequencing techniques can be used to identify polypeptides expressed by senescent cells.

The invention will be further described in the following examples, which do not limit the scope of the invention described in the claims.

### EXAMPLES

### Example 1—Materials and Methods

p21-Cre mouse model generation: A 7 kb DNA fragment, containing a 3225 bp mouse p21 promoter fragment followed by a nucleotide sequence encoding Cre fused to a tamoxifen-inducible estrogen receptor (ER^{T2}) domain, was synthesized by GenScript (Piscataway, N.J.). An IRES followed by an open reading frame (ORF) coding for enhanced green fluorescent protein (EGFP) also was added into the transgene. The synthesized fragment (p21-ER-Cre) was subcloned into vector pBT378 for generating p21-Cre mice through Integrase-mediated transgenesis (IMT). Briefly, the p21-ER-Cre-pBT378 construct was microinjected into the pronucleus of recipient zygotes containing attP sites in the Hipp11 locus, which has a high recombination rate and results in stable expression of a single copy of the transgene. Site-specific recombination occurred between the attB sites in the plasmid and attP sites in the H11 genomic locus. Positive embryonic stem (ES) cell clones were then implanted into C57BL/6 females. A positive founder mouse was confirmed by PCR using a set of primers flanking the recombined 5′- and 3′-attP/attB sites in the H11 locus, as well as sets of specific primers that amplify unique sequences within the transgene.

Mouse models and drug treatments: All mice were maintained in a pathogen-free facility at 23-24° C. under a 12-hour light, 12-hour dark regimen with free access to a RCD (Teklad global 18% protein, Envigo #2918, Indianapolis, Ind.), or a 60% (by calories) HFD (D12492, irradiated; Research Diets, New Brunswick, N.J.) and water. For tamoxifen treatment, tamoxifen (Sigma-Aldrich, St Louis, Mo.) was dissolved in corn oil, and was administrated to mice by intraperitoneal (i.p.) injection once daily (2 mg per mouse) for two consecutive days. For DOXO treatment, DOXO (Sigma-Aldrich) was given to mice (20 mg/kg) once by i.p. injection. Tamoxifen was given in one dose at the same time of DOXO administration, and one more dose was given 16 hours after DOXO administration. Floxed tdTomato mice (#007914), floxed LUC mice (#005125), floxed DTA mice (#009669), CAG-Cre mice (#004682), and Rela^{fl/fl} mice (#024342) were purchased from the Jackson Laboratory (Bar Harbor, Me.).

Single cell RNA-seq analysis: Single cell RNA-seq data was collected from the Tabula Muris Senis single cell transcriptomic atlas for aged mice (Tabula Muris, Nature 583:590-595, 2020). Data were visualized using the provided browser based platform available at tabula-muris-senis.ds.czbiohub.org. In brief, single cell RNA seq data were examined from visceral adipose tissue (fluorescence-assisted cell sorting (FACS) method), liver (droplet method), and heart (droplet method). To account for differences in animal ages available for each tissue, all available data from animals 18 months or older was included in the analysis (Fat: 18 and 24 months; Liver: 18, 21, 24, 30 months; Heart: 18, 21, 24, 30 months). Expression profiles were then captured for p21 and p16 in each tissue.

Tissue dissociation: Visceral fat and liver tissues were minced with scissors, and digested in PBS containing 1 mg/ml type II collagenase (Sigma-Aldrich) and 10 μg/ml DNase I (Sigma-Aldrich) at 37° C. for 1 hour. After digestion, SVF cells were separated from visceral fat. SVF cells and liver cells were washed with phosphate buffered saline (PBS)/2% fetal bovine serum (FBS) and filtered through a 100 μm cell strainer. Cells were then incubated with ammonium-chloride-potassium (ACK) lysing buffer (Thermo Fisher Scientific, Waltham, Mass.) at room temperature for 10 minutes to remove red blood cells. Cells were washed with PBS/2% FBS for further experiments.

SA-β galactosidase staining. SA-β gal staining was assayed as described elsewhere (Xu et al., Proc Natl Acad Sci USA 112:E6301-6310, 2015; and Biran et al., Aging Cell 16:661-671, 2017). Briefly, purified SVF cells were fixed in 4% paraformaldehyde (PFA, Thermo Fisher Scientific) for 15 minutes at room temperature. Cells were washed with PBS, then incubated with SA-β gal staining solution containing 1 mg/ml X-gal, 40 mM citric acid/sodium phosphate at pH 6.0, 5 mM potassium ferrocyanide, 5 mM potassium ferricyanide, 150 mM NaCl, and 2 mM MgCl₂ at 37° C. in a humidified chamber, and protected from light. After 16 hours of incubation, cells were washed in ice-cold PBS to stop the enzymatic reaction and resuspended in PBS buffer for ImageStreamX analysis.

EdU staining. Mice fed HFD for 5 months were treated with 1 mg/kg 5-ethynyl-2′-deoxyuridine (EdU, Cayman Chemical, Ann Arbor, Mich.) via intraperitoneal injection. After 20 hours, SVF cells were isolated and fixed in 4% PFA for 15 minutes at room temperature. After being washed with PBS/1% BSA, cells were permeabilized by 0.3% Triton X-100, and stained with 100 mM Tris, 2 mM CuSO₄ (Sigma-Aldrich), 10 mM ascorbic acid, 2 μM Alexa Fluor 647 azide for 30 minutes at room temperature. Cells were washed with PBS/1% BSA twice, incubated with 0.1 μg/ml DAPI (Sigma-Aldrich) for 5 minutes, and analyzed by ImageStreamX.

Imaging flow cytometry analysis: SVF cells stained with SA-β gal and EdU were imaged by ImageStreamX Mark II (Amnis, Seattle, Wash.) and analyzed by IDEAS 6.2 software (Amnis). To focus cells, samples were gated using gradient RMS values of brightfield channel. Cells were further gated using brightfield area and aspect ratio to select single cells. GFP− and GFP+ SVF cells were measured for cell areas using brightfield area. Mean pixel of brightfield was used to calculate SA-β gal intensity in GFP− and GFP+ SVF cells as described elsewhere (Biran et al., supra). To measure EdU, the intensity of APC channel was compared between GFP− and GFP+ SVF cells.

Cell culture: Ear fibroblasts were isolated as described elsewhere (Xu et al., J Gerontol A Biol Sci Med Sci 72:780-785, 2017). To induce senescence, ear fibroblasts were treated with 0.5 μM DOXO for 24 hours, and considered to be senescent 10 days after.

Immunofluorescence staining: SVF cells were fixed in 4% PFA for 15 minutes at room temperature. Cells were then washed with PBS prior to a 10 minute incubation in permeabilization buffer (0.2% Triton X-100, 1% BSA in PBS). Next, the cells were washed with PBS and blocked for 1 hour in PBS/1% BSA solution. After blocking, cells were incubated with anti-Lamin B1 primary antibody (Proteintech, Rosemont, Ill.) 1:100 dilution in PBS/1% BSA overnight at 4° C. The next day, cells were washed with PBS and incubated for 1 hour at room temperature with Alexa Fluor 647-conjugated anti-rabbit secondary antibody (Thermo Fisher Scientific) diluted 1:200 in PBS/1% BSA. Stained cells were then washed with PBS and analyzed by flow cytometry.

Flow cytometry analysis: SVF cells and liver cells were isolated from visceral fat and liver tissues, and washed with PBS/2% FBS. Cells were stained with 0.1 μg/ml of DAPI for 5 minutes and detected by BD LSR II flow cytometer (BD Biosciences, San Jose, Calif.). Data analysis was performed using FlowJo V10.7 software.

FACS: SVF cells were isolated from mice fed HFD for 5 months, and were sorted into GFP− and GFP+ populations through BD FACSAria II flow cytometer (BD Biosciences). Sorted cells were used to detect p21, p16 and SASP expression.

Bioluminescence imaging: Mice were anesthetized with isoflurane gas (Piramal Critical Care, Bethlehem, Pa.) and injected intraperitoneally with 3 mg D-luciferin (Gold Biotechnology, St. Louis, Mo.) in 200 μl PBS. Five (5) minutes after injection, mice were placed in an IVIS spectrum in vivo imaging system (PerkinElmer, Waltham, Mass.) and bioluminescence images were subsequently captured with a 3 minute exposure time. The region of interest (ROI) was manually selected in a consistent way for individual mice in the same cohort. Bioluminescent signal of ROI was calculated using the Living Image 4.5.5 software (PerkinElmer).

Histological analysis: Visceral fat, liver, intestine, brain, heart, and muscle tissues were fixed with 4% PFA overnight at 4° C. After being washed with PBS, tissues were transferred to 30% sucrose (Sigma-Aldrich), incubated overnight at 4° C., and embedded in OCT compound (Thermo Fisher Scientific). Liver, intestine, brain, heart, muscle tissues in OCT blocks were cut into 6 μm thickness sections on a Leica CM3050 S cryostat, and visceral fat tissues were cut into 10 μm thick sections. Nuclei were counterstained with Hoechst 33342 (Thermo Fisher Scientific) and imaged on a fluorescence microscope (Zeiss, Jena, Germany). To quantify tdTomato+ cells, three sections from each mouse were scanned and then counted using ImageJ software.

Physical function measurements: Physical function measurements were performed as described elsewhere (Xu et al., Nature Med 24:1246-1256, 2018). To test muscle strength and neuromuscular function, maximal walking speed, grip strength and grid hanging test were performed in aged mice. Maximal walking speed was tested with an accelerating 4 lane RotaRod system (Columbus Instruments, Columbus, Ohio). Mice were trained on the RotaRod for 2 consecutive days at a constant speed of 4 r.p.m. for 300 seconds. The day after the last day of training, mice were acclimatized to the testing room for 30 minutes. RotaRod test was started at 4 r.p.m. and accelerated from 4 to 47.2 r.p.m. in 6 minutes. The speed was recorded when the mouse fell off, and the average of 4 trials were calculated and normalized to the baseline speed. Forelimb grip strength (g) was assessed by a grip strength test meter (Bioseb, France). Results were averaged over 10 trials. A grid hanging test was performed on a grid placed onto a holding apparatus at a 35 cm distance from the floor, using a soft pad to avoid injuries. Hanging time was recorded when the mouse fell off, and the average of 3 trials were calculated and normalized to body weight as hanging duration (seconds)×body weight (g). A threshold at 7 minutes was set to consider the maximum hanging time.

Comprehensive laboratory animal monitoring system (CLAMS): Daily activity, food and water intake, metabolic performance, temperature were measured for the 23 month-old PL and PLD mice on a CLAMS system equipped with an Oxymax open-circuit calorimeter (Columbus Instruments, Columbus, Ohio). Physiological and behavioral parameters for individual mice were monitored over a 24 hour period (12 hour light/dark cycle), and results were analyzed by CLAX software (Columbus Instruments).

DNA preparation and PCR: Rela and P-Rela mice were sacrificed 24 hours after tamoxifen injection for 3 doses. Genomic DNA was extracted by lysing SVF cells in an alkaline reagent containing 25 mM NaOH and 0.2 mM EDTA at 94° C. for 2 hours and neutralizing with an equal volume of 40 mM Tris-HCl (pH 7.4), followed by purifying with isopropanol and 75% ice-cold ethanol (Sigma-Aldrich). An equal amount of DNA for each sample was adopted to react with GoTaq Green Master Mix (Promega, Madison, Wis.) and the indicated genotyping primers, respectively. PCR products were separated on a 2% agarose gel with SYBR safe DNA gel stain (Thermo Fisher Scientific) running in TAE buffer (Bio-Rad, Hercules, Calif.) and visualized using the ChemiDoc MP imaging system (Bio-Rad). All primers were purchased from Integrated DNA Technologies (IDT, Coralville, Iowa). Primer sequences are shown in TABLE 1.

TABLE 1

| Primer | Sequence 5′ to 3′ | SEQ ID NO: |
|---|---|---|
| Cre-F | ACCAGCCAGCTATCAACTCG | 11 |
| Cre-R | TTACATTGGTCCAGCCACC | 12 |
| SH176-F | TGGAGGAGGACAAACTGGTCAC | 13 |
| SH178-R | TTGTTCCCTTTCTGCTTCATCTTGC | 14 |
| BT436-R | ATCAACTACCGCCACCTCGAC | 15 |
| Rela-F | GACACGCTGAACTTGTGGCCGTTTA | 16 |
| Rela-WT-R | TATCATGTCTGGATCAATTCATAAC | 17 |
| Rela-Cut-R | TTTCGACCTGCAGCCAATAAGCT | 18 |

RNA extraction and real-time PCR: Cells were collected, lysed in TRIzol reagent (Thermo Fisher Scientific), and extracted with chloroform, isopropanol, and 75% ice cold ethanol (Sigma-Aldrich). RNA was dissolved in RNase free water and reverse transcribed to cDNA with M-MLV reverse transcriptase kit (Thermo Fisher Scientific). Quantitative real-time PCR was conducted in four or five replicates using PerfeCTa FastMix II (Quantabio, Beverly, Mass.) on the CFX96 Real-Time PCR detection system (Bio-Rad). The relative mRNA level of target genes was normalized to TATA-binding protein (Tbp) and calculated via the 2^{-ΔΔCT} method. Probes and primers for Tbp, p21, p16,116, Cxcl1, Ccl2 and Rela were purchased from Integrated DNA Technologies (IDT, Coralville, Iowa):

TABLE 2

| Target | IDT Id |
|---|---|
| Tbp | Mm.PT.39a.22214839 |
| p21 | Mm.PT.58.17125846 |
| p16 | Mm.PT.58.8388138 |
| Il6 | Mm.PT.58.10005566 |
| Rela | Mm.PT.58.29633634 |
| Ccl2 | Mm.PT.58.42151692 |
| Cxcl1 | Mm.PT.58.42076891 |

### Example 2—p16^{high} and p21^{high} Cells Represent Two Distinct Cell Populations in Aged Tissues

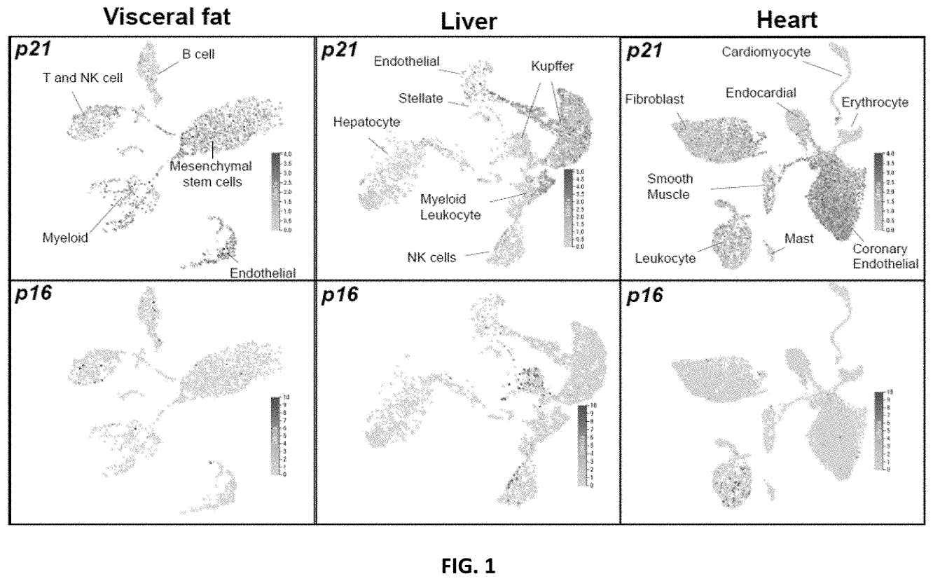

Senescent cells are highly heterogeneous in their biological properties, tissue distribution and responses to varied therapies (Roy et al., Cell 183:1143-1146, 2020). To explore the possible overlap of p16^{high} cells and p21^{high} cells or their respective population diversity, a single cell transcriptomic (SCT) atlas database (Tabula Muris Senis; Tabula Muris, Nature 583:590-595, 2020) was used. This database includes transcriptomic data from a range of tissues in 18-30 month-old mice. p21 and p16 expression levels were visualized using the browser-based interactive platform (tabula-muris-senis.ds.czbiohub.org/). In aged visceral fat, p21^{high} cells are mainly endothelial cells, mesenchymal stem cells, and myeloid cells, while p16^{high} cells are scarce. In liver, p21^{high} cells are mainly Kupffer cells and myeloid cells, while p16^{high} cells are mainly natural killer (NK) cells and a different population of Kupffer cells. In heart, p21^{high} cells are mainly endothelial cells, while p16^{high} cells are mainly leukocytes (FIG. 1). Thus, p16^{high} cells and p21^{high} cells are indeed two distinct populations, at least in aged tissues, emphasizing the need for models targeting p21^{high} cells.

### Example 3—Generation of p21-Cre Mouse Model

To generate the p21-Cre mouse model, a 7 kb DNA fragment (FIG. 2A) was synthesized containing a 3225 bp mouse p21 promoter fragment followed by a nucleotide sequence encoding Cre recombinase (Cre) fused to a tamoxifen-inducible estrogen receptor (ER^{T2}) domain (Feil et al., Biochem Biophys Res Commun 237:752-757, 1997). Cre-ER^{T2} is normally retained in the cytoplasm (inactive) in the absence of an inducer. Upon addition of tamoxifen or 4-hydroxytamoxifen (4-OH), the Cre-ER^{T2} translocates to the nucleus where it acts preferentially on loxP sites. An internal ribosome entry site (IRES) followed by an open reading frame (ORF) coding for enhanced green fluorescent protein (GFP) was added to the construct to facilitate detection of p21^{high} cells. The 3225 bp mouse p21 promoter fragment is well conserved between human and mouse, and contains three p53-binding sites that are responsive to DNA damage (el-Deiry et al., Cancer Res 55:2910-2919, 1995). Integrase-mediated transgenesis (IMT) (Tasic et al., Proc Natl Acad Sci USA 108:7902-7907, 2011) was used to generate the p21-Cre transgenic mice. The synthesized fragment (p21-Cre) was first subcloned into the vector pBT378 containing two attB sites, and the vector was microinjected into the pronucleus of recipient zygotes containing attP sites in the Hipp11 (H11, chromosome 11) locus, which has a high recombination rate and results in stable expression of a single copy of the transgene (Hippenmeyer et al., Neuron 68:695-709, 2010). Site-specific recombination occurred between the attB sites in the construct and the attP sites in the H11 genomic locus, leading to insertion of the p21-Cre transgene into the H11 locus.

An advantage of this site-specific transgenic approach using the H11 locus is that it was less likely to interfere with or disrupt any endogenous gene, as opposed to the use of random insertion or insertion into targeting gene locus. In addition, this approach allowed for the design of genotyping primers to distinguish +/+, p21-Cre/+, and p21-Cre/p21-Cre genotypes (FIG. 2B). As shown in FIG. 2C, two different sets of genotyping primers were validated. For set 1, SH176-F (SEQ ID NO:13) and SH178-R (SEQ ID NO:14) generated a 326 bp band for the wild type (WT) allele, while Cre-F (SEQ ID NO:11) and Cre-R (SEQ ID NO:12) generated a 200 bp band for the p21-Cre transgene allele. For set 2, SH176-F (SEQ ID NO:13) and SH178-R (SEQ ID NO:14) generated a 326 bp band for the WT allele, while SH176-F (SEQ ID NO:13) and BT436-R (SEQ ID NO:15) generated a 260 bp band for the p21-Cre transgene allele. Primer sequences are shown in TABLE 1.

### Example 4—Whole-Body Live Imaging of p21^{high} Cells In Vivo

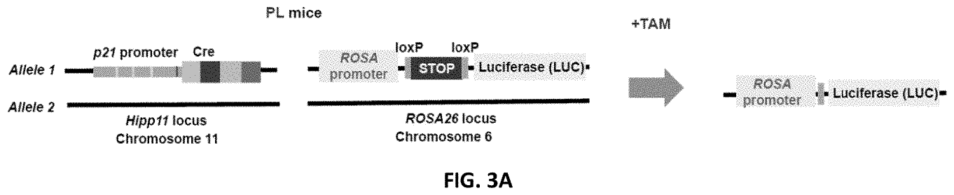

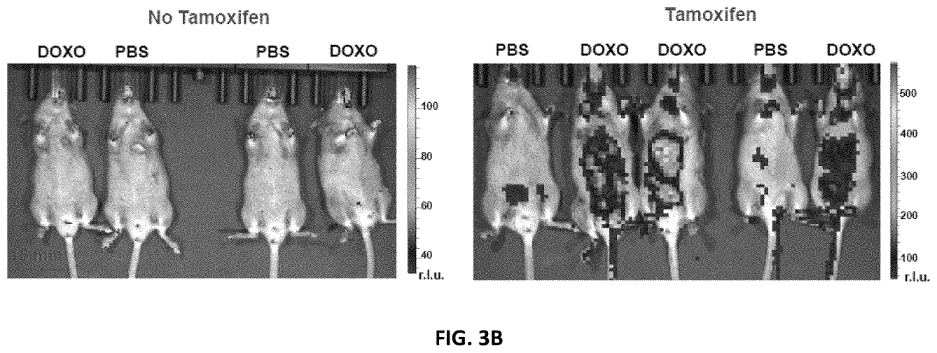

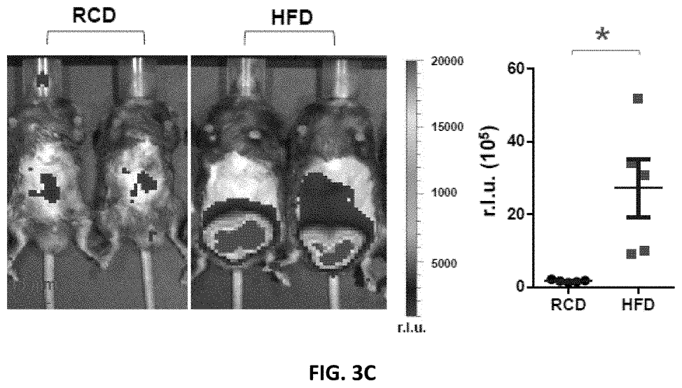

To validate function of the transgene, the p21-Cre mouse was crossed with floxed knock-in firefly luciferase (LUC) mice (Safran et al., Mol Imaging 2:297-302, 2003). Floxed knock-in LUC mice contain a loxP-flanked STOP fragment between the Gt(ROSA)26Sor (ROSA) promoter and LUC, which prevents LUC expression without the presence of Cre (FIG. 3A). The p21-Cre/+; LUC/+(PL) mice resulting from the cross contained one copy of p21-Cre in chromosome 11 and one copy of LUC in chromosome 6. This PL mouse model allowed for detection of p21^{high} cells in live mice through bioluminescence imaging (BLI) in a temporal manner. DOXO is a potent DNA damaging agent that can induce p21 expression (Tinkum et al., Mol Cell Biol 31:3759-3772, 2011) and accumulation of p21^{high} cells. PL mice were treated with DOXO and tested to determine whether p21^{high} cells could be detected in vivo. Little signal was observed in either the DOXO- or PBS-treated groups without tamoxifen treatment (FIG. 3B, left panel), indicating that Cre or STOP fragment leakage was minimal. After 2 tamoxifen treatments to induce Cre and subsequent LUC activity in p21^{high} cells, DOXO-treated PL mice had more BLI signal compared to PBS-treated ones (FIG. 3B, right panel), suggesting that the transgene worked properly.

Metabolic stress and obesity can induce cellular senescence (Xu et al., Nature Med 24:1246-1256, 2018) and p21 expression (Schafer et al., Diabetes 65:1606-1615, 2016). Studies were conducted to examine whether p21^{high} cells could be detected in PL mice under metabolic stress. PL mice were fed HFD for 4 months and then treated with two doses of tamoxifen. HFD led to significantly increased BLI signals in PL mice when compared to normal chow diet (NCD), indicating accumulation of p21^{high} cells with obesity (FIG. 3C). Additional studies were done to investigate whether p21^{high} cells are induced with aging. Young (3-month-old) and old (23-month-old) PL mice were treated with two doses of tamoxifen and subjected to BLI, revealing that BLI signals were significantly higher in old PL mice than in young mice (FIG. 3D). Thus, all three conditions (chemotherapy drugs, obesity, and aging) induced p21^{high} cells, which were successfully monitored by BLI in live mice.

### Example 5—p21^{high} Cells Accumulate in Various Tissues with Aging

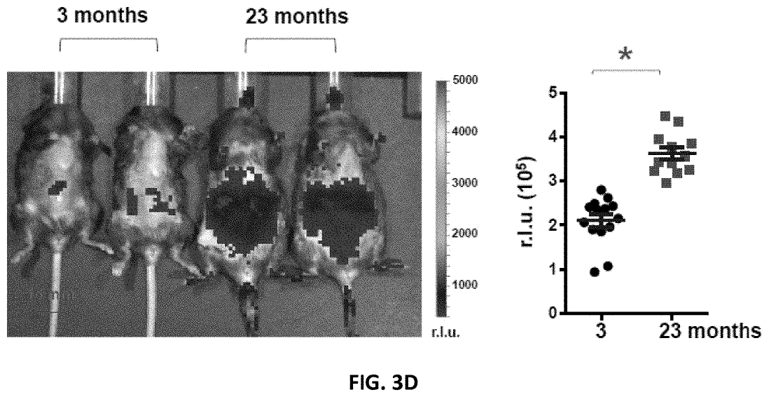

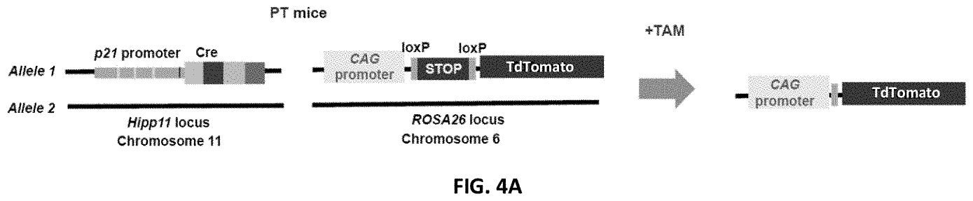

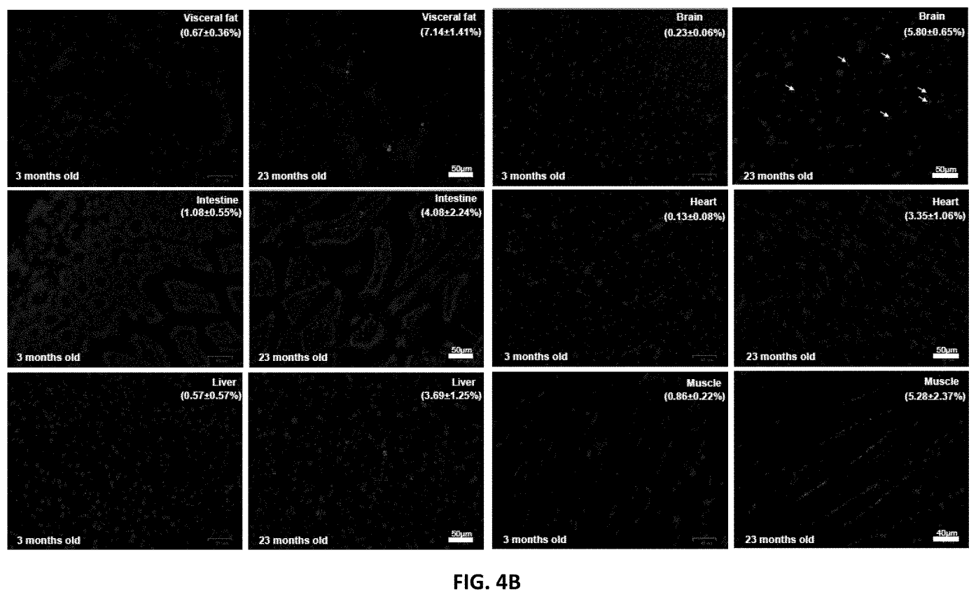

To image p21^{high} cells using fluorescence in vivo at the tissue level, p21-Cre mice were crossed with floxed knock-in tdTomato mice (Madisen et al., Nat Neurosci 13:133-140, 2010), which contain a loxP-flanked STOP fragment between the CMV early enhancer/chicken p actin (CAG) promoter and tdTomato, yielding p21-Cre/+; tdTomato/+ (PT) mice (FIG. 4A). Compared to the GFP included in the transgene (driven by the p21 promoter), tdTomato (driven by the strong CAG promoter) generates a much brighter red fluorescent signal with less confounding by an autofluorescent background, which makes it highly suitable for in vivo fluorescence imaging. Young (3-month-old) and old (23-month-old) PT mice were treated with two doses of tamoxifen and various tissues were collected for fluorescence imaging. Most of the tissues from old PT mice contained more tdTomato+p21^{high} cells, including visceral fat, brain, intestine, heart, liver, and skeletal muscle (FIG. 4B). The percentages of p21^{high} cells in these old tissues ranged from 1.5 to 10%—comparable to the percent of senescent cells in aged tissues. No reliably tdTomato+ cells were seen in aged kidneys due to high autofluorescence, and very few tdTomato+ cells were observed in the aged lung. Importantly, very few tdTomato+ cells were seen in tissues collected from young and healthy PT mice, indicating that the p21-Cre mouse model might only target age- or disease-specific cells, such as senescent cells, without affecting most cells in young mice.

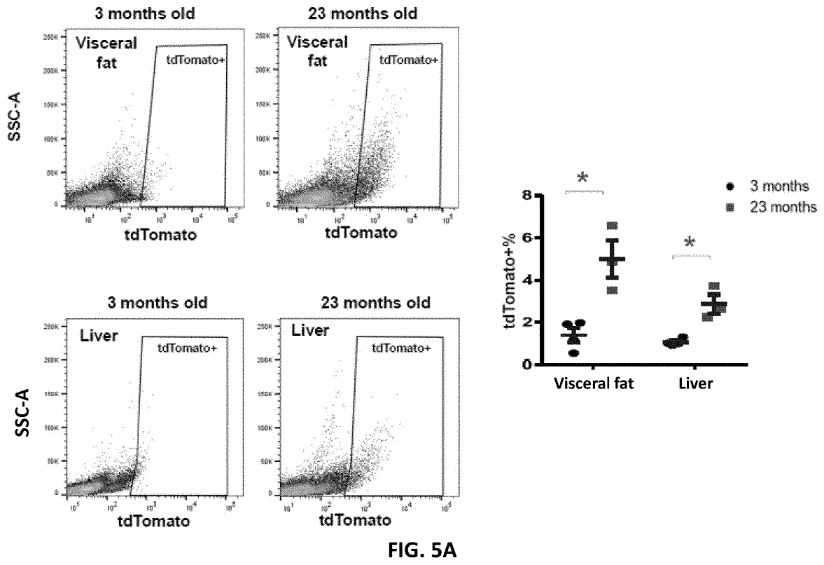

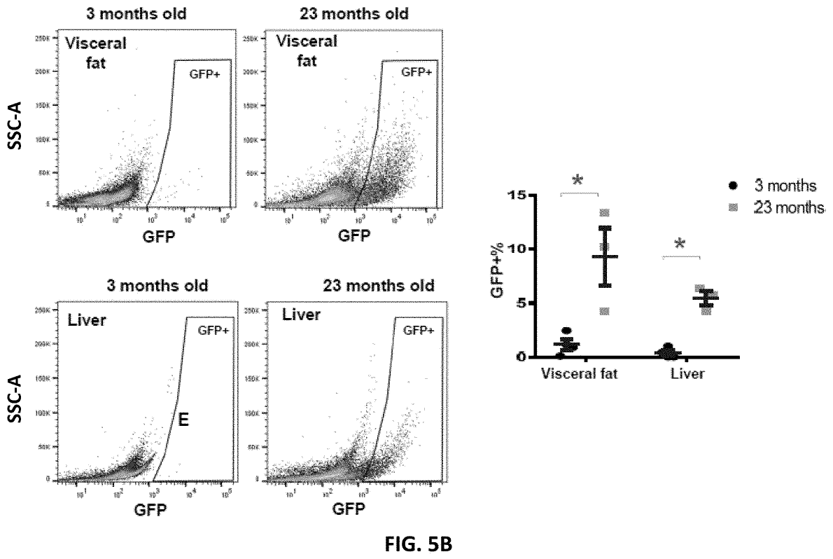

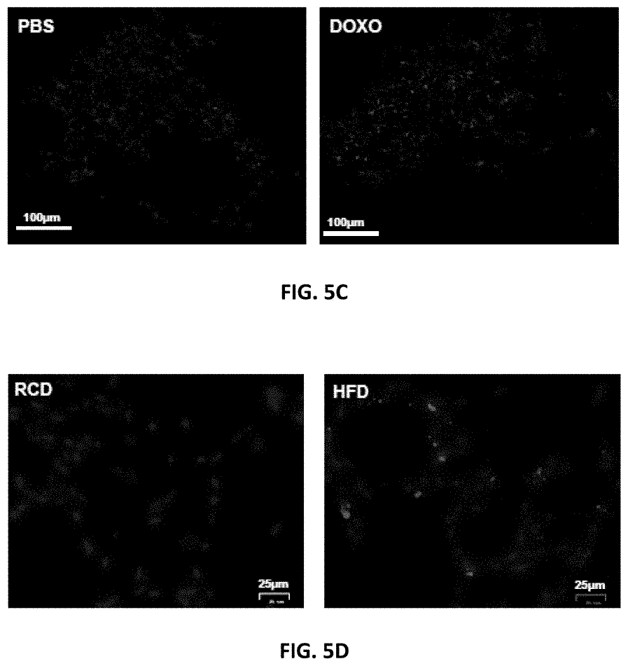

p21^{high} cells can also be detected by flow cytometry. Consistent with the fluorescence imaging, flow cytometry analysis revealed that the percentage of tdTomato+ p21^{high} cells in visceral fat and liver was higher in old mice than in young PT mice (FIG. 5A). Flow cytometry analysis also was carried out using GFP, yielding similar results (FIG. 5B), indicating that both tdTomato and GFP can be used to detect p21^{high} cells with flow cytometry. In addition to aging, both DOXO treatment and HFD induced accumulation of tdTomato+p21^{high} cells in visceral fat (FIGS. 5C and 5D), similar to the findings using BLI (FIGS. 3B and 3C). These results suggested that p21^{high} cells specifically accumulate in various tissues with aging or other conditions and can be precisely detected in the mouse models provided herein.

### Example 5—p21^{high} Cells Exhibit Features of Cellular Senescence

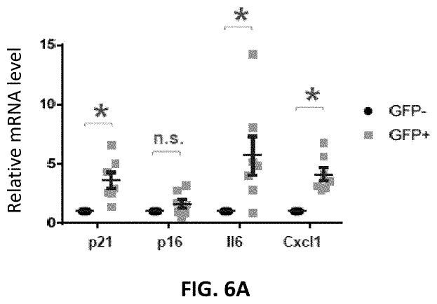

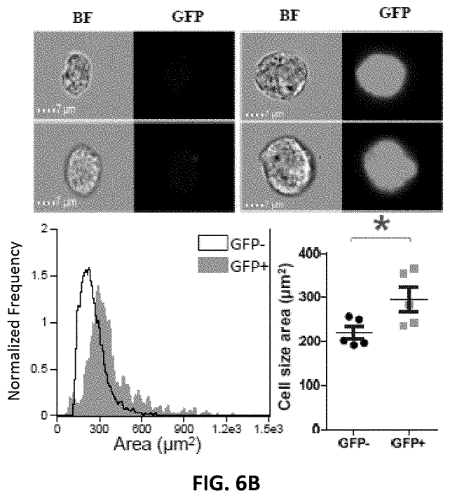

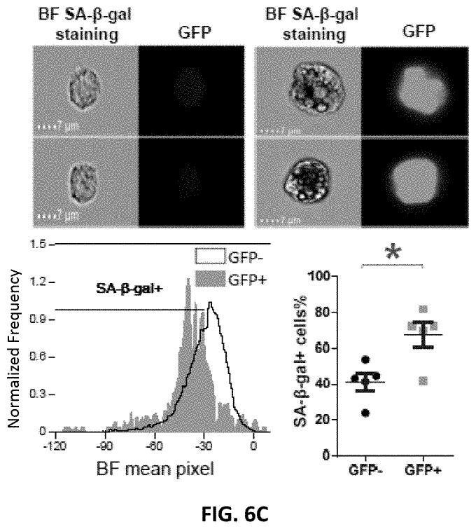

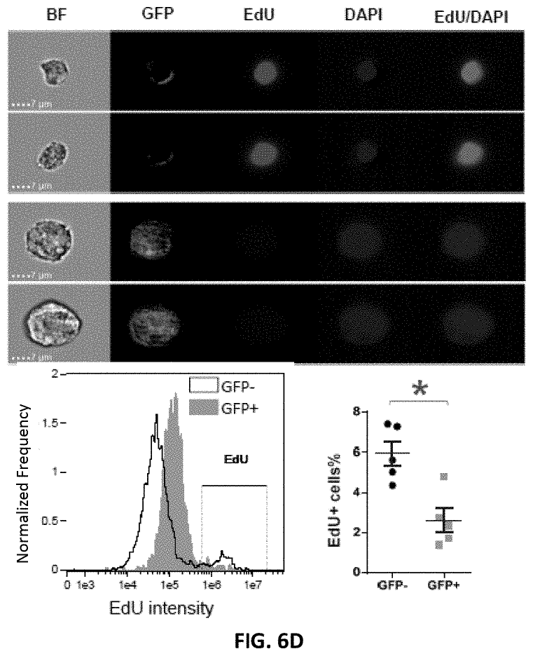

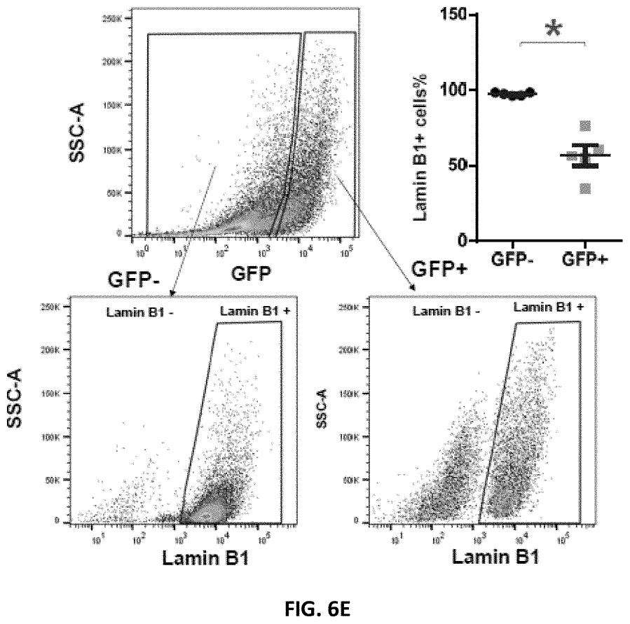

p21^{high} adipose-derived mesenchymal stem cells (ADSCs) have been shown to accumulate in aged mice (Wang et al., Aging Cell e13106, 2020). Single cell transcriptomics revealed that these naturally occurring p21^{high} cells appear to exhibit altered pathways commonly observed in senescent cells, including Senescent Cell Anti-Apoptotic Pathways (SCAPs; increased cell survival and decreased apoptosis) and NF-κB, IL6/JAK, mTOR, FOXO, and HMGB1 pathways. Studies were conducted to further characterize p21^{high} cells using the p21-Cre mouse model in vivo. PL mice were fed a HFD for 5 months to induce p21^{high} cells in visceral fat (FIG. 5D). The stromal vascular fraction (SVF) was isolated from fat, and GFP+ p21^{high} cells and GFP− non-p21^{high} cells were separated using fluorescence-activated cell sorting (FACS). Compared to GFP− cells, GFP+ p21^{high} cells expressed 4-fold higher levels of p21, as well as 6-fold and 4-fold higher level of two major SASP components (Il6 and Cxcl1, respectively). Notably, p16 mRNA levels were not statistically different between these two cell populations (FIG. 6A). Imaging flow cytometry (ImageStreamX) technology, which combines flow cytometry with high content image analysis, was leveraged to further characterize GFP+ p21^{high} cells at the single cell level. Enlarged size, senescence-associated-s-galactosidase (SA-β-gal), and proliferation arrest are three major features of senescent cells both in 10 vitro and in vivo (Di Micco et al., Nat Rev Mol Cell Biol, 2020). Analysis of bright field (BF) images showed that GFP+ cells exhibited a larger cell size compared to GFP− cells (FIG. 6B). Next, SVF was stained with SA-β-gal staining buffer and SA-β-gal+ cells were quantified using a method described elsewhere (Biran et al., Aging Cell 16:661-671, 2017). More than 60% of GFP+ cells were SA-β-gal+, compared to 40% of GFP− cells (FIG. 6C). A thymidine analog, 5-ethyl-2′-deoxyuridine (EdU), was injected into the mice, and incorporation of EdU was assessed using imaging flow cytometry 20 hours later. GFP− cells had more EdU+ cells (6%) than GFP+ cells (2%; FIG. 6D), suggesting that GFP+ cells had reduced proliferation rates. Loss of Lamin B1 is another marker for senescent cells (Freund et al., Mol Biol Cell 23:2066-2075, 2012). By flow cytometry analysis, most GFP− cells (97%) were found to express Lamin B1, while Lamin B1 was absent in 43% of GFP+ cells (FIG. 6E). Taken together, these studies demonstrated that p21^{high} cells exhibited characteristics typical of senescent cells, including higher expression of p21, the SASP, enlarged size, SA-β-gal-positivity, reduced proliferation, loss of Lamin B1, and a number of altered senescence-associated pathways.

### Example 7—Inducible Elimination of p21^{high} Cells Using Diphtheria Toxin A

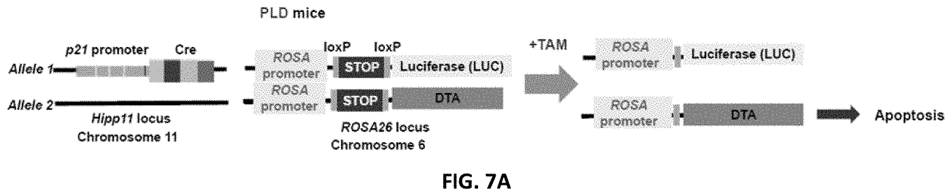

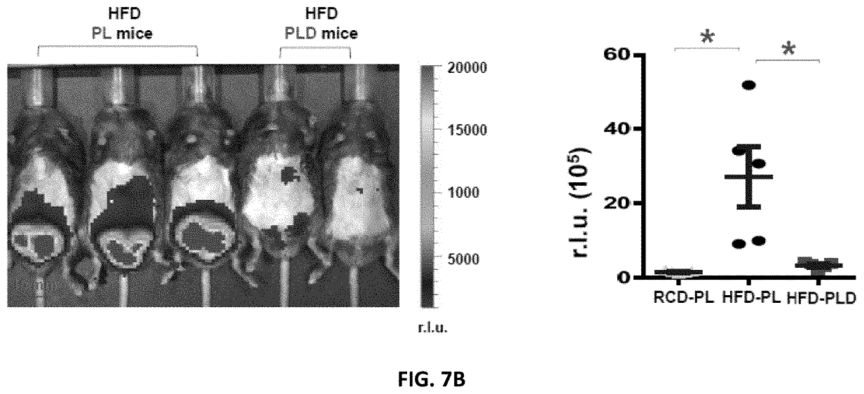

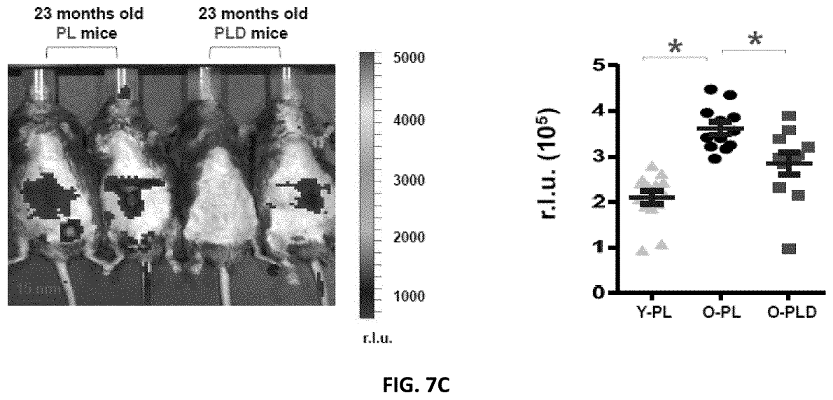

To enable elimination of p21^{high} cells in a temporal manner in vivo, PL mice were crossed with floxed diphtheria toxin A (DTA) mice (Voehringer et al., J Immunol 180:4742-4753, 2008), which contain a floxed-STOP cassette followed by DTA driven by the ROSA promoter. p21-Cre/+; LUC/DTA (PLD) mice were generated (FIG. 7A), which contain one copy of p21-Cre at the Hipp11 locus, one copy of LUC at the ROSA locus, and one copy of DTA at the ROSA locus. Diphtheria toxin has two subunits, A and B. Subunit B is responsible for binding the receptor and internalization of DTA. Once inside cells, DTA inhibits eukaryotic translation elongation factor 2 (eEF2), leading to protein translation block and ensuing apoptosis (Oppenheimer and Bodley, J Biol Chem 256:8579-8581, 1981). Since PLD mice have only DTA without subunit B, it was theoretically unlikely that DTA leakage from p21^{high} cells (if any) would enter nearby non-p21^{high} cells and kill them. Thus, the PLD mice allowed for specific killing (by DTA) and monitoring p21^{high} cells (by LUC) in vivo. To validate clearance, both PL and PLD mice were fed a HFD for three months. After two doses of tamoxifen treatment, HFD-fed PL mice had high BLI signals, while the BLI signals were much lower in PLD mice (FIG. 7B), demonstrating successful clearance of p21^{high} cells from obese mice. Two doses of tamoxifen treatment also reduced the BLI signal in 23-month-old PLD mice compared to PL mice (FIG. 7C), suggesting that p21^{high} cells can be eliminated using the model provided herein with aging as well.

### Example 8—Genetic Inhibition of the SASP in p21^{high} Cells In Vivo

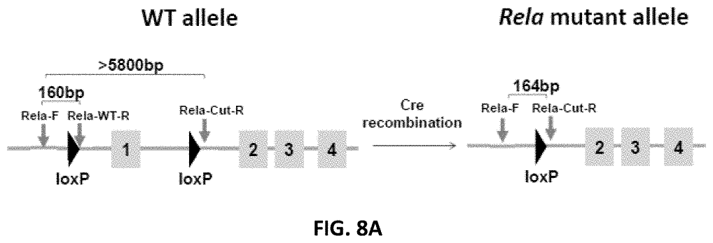

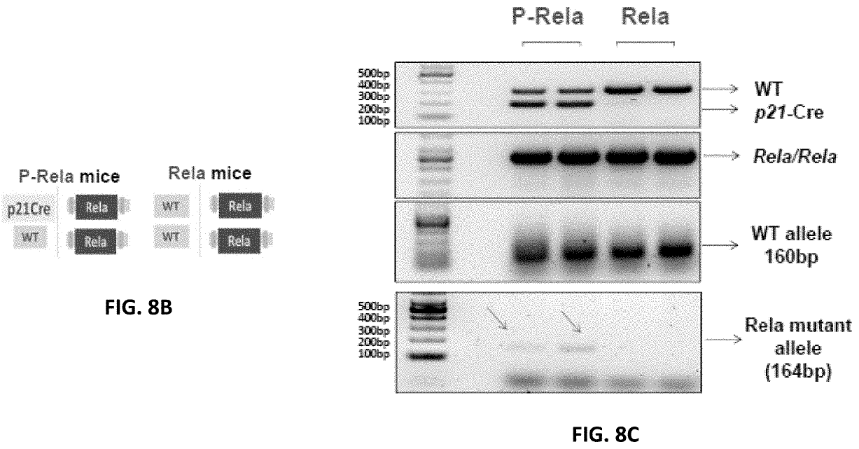

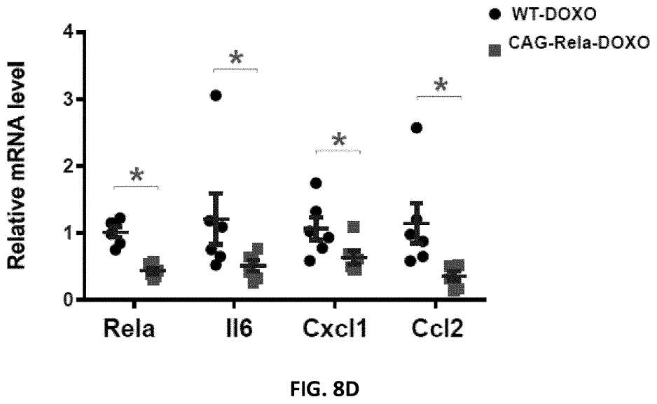

The role of senescent cells has been extensively examined across a range of pathological conditions, but the underlying mechanisms have rarely been investigated in vivo. The SASP is thought to be one of the major mechanisms responsible for the harmful effects of senescent cells (Tchkonia et al., J Clin Invest 123:966-972, 2013). Studies were conducted to determine the effects of the p21-Cre mouse model provided herein on the SASP exclusively in p21^{high} cells. The NF-κB pathway serves as a master regulator of the SASP (Chien et al., Genes Dev 25:2125-2136, 2011), and Rela (v-rel reticuloendotheliosis viral oncogene homolog A, or p65) is a crucial subunit for NF-κB activation (Chen and Greene, Nat Rev Mol Cell Biol 5:392-401, 2004). Both Rela and the NF-κB pathway are highly activated in p21^{high} preadipocytes isolated from aged mice (Wang et al., Aging Cell e13106, 2020). To inactivate the NF-κB pathway in p21^{high} cells, p21-Cre mice were crossed with floxed Rela mice (Heise et al., J Exp Med 211:2103-2118, 2014), in which exon 1 of the Rela gene is flanked by loxP sites (FIG. 8A). p21Cre/+; Rela^{fl/fl} (P-Rela) mice containing one copy of p21-Cre and two copies of floxed Rela were generated, as were +/+; Rela^{fl/fl} (Rela) mice containing only two copies of floxed Rela (FIG. 8B). These mice were fed a HFD for three months to induce p21^{high} cells. SVF cells were collected from visceral fat after two doses of tamoxifen administration to allow Rela mutation detection. Primers were designed that generated a 164 bp Rela mutant band and a 160 bp WT band. Rela mutant bands were detected only in P-Rela SVF cells, while WT bands were detected in both P-Rela and Rela SVF cells (FIG. 8C). The Rela mutation was confirmed by sequencing the Rela mutant bands. In addition, Rela mutant bands were much fainter than WT bands, and strong WT bands were also observed in P-Rela SVF cells, indicating that only a small percent of P-Rela SVF cells (p21^{high} cells) had the Rela mutation.

Next, studies were conducted to determine whether the Rela mutation led to SASP inhibition. CAG-Cre mice (Hayashi and McMahon, Devel Biol 244:305-318, 2002), which carry a constitutively active CAG promoter driving Cre, were crossed with floxed Rela mice to generate CAG-Cre/+; Rela^{fl/fl} (CAG-Rela) mice. Ear fibroblasts were isolated from these mice and senescence was induced using DOXO. After 4-OH treatment to induce Cre, expression levels of Rela and several key SASP components were observed to be reduced by 40-70% in senescent CAG-Rela cells compared to senescent WT cells (FIG. 8D). Thus, the SASP of senescent cells can be genetically suppressed in the mouse models provided herein.

### Example 9—Intermittent Clearance of p21^{high} Cells Improves Physical Function in Old Mice

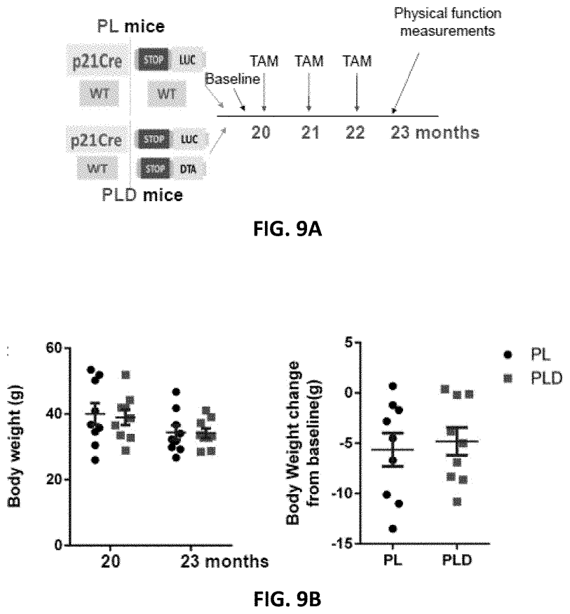

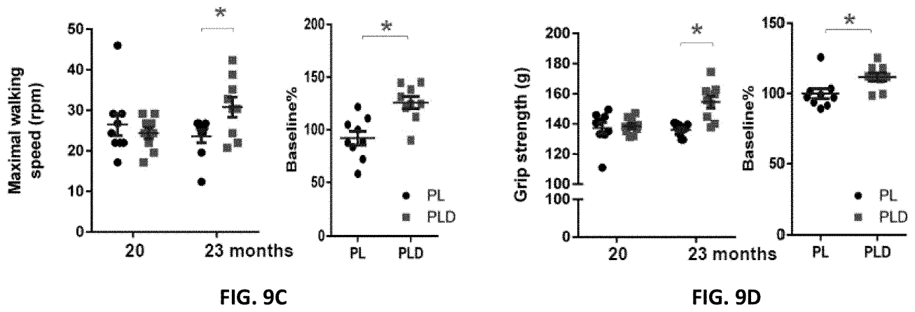

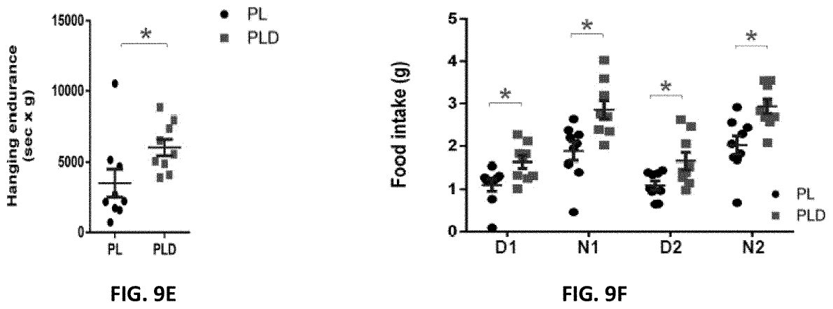

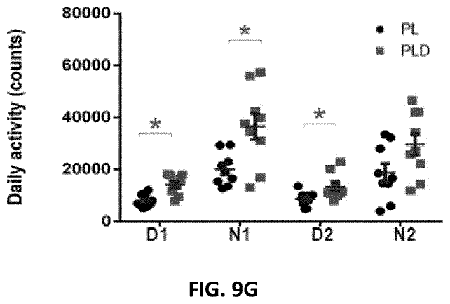

Physical function typically declines with aging, leading to physical frailty, compromised systemic homeostasis, and increased vulnerability to stresses (Fried et al., Nature Aging 1:36-46, 2021). To investigate whether p21^{high} cells play a causal role in physical frailty with aging, 20-month-old PL and PLD mice were treated with two doses of tamoxifen per month for three months (FIG. 9A) to eliminate p21^{high} cells in PLD mice (FIG. 7C). Physical frailty was assessed following the frailty criteria widely used in clinic practice (Fried et al., J Gerontol A Biol Sci Med Sci 56:M146-156, 2001; and Justice et al., EBioMedicine 40:554-563, 2019), including weight loss, walking speed, grip strength, physical endurance, food intake, and daily activity. No difference was observed in body weight, maximal walking speed, or grip strength between 20-month-old PL and PLD mice before tamoxifen treatment. After three months of intermittent tamoxifen administration (a total of 6 doses), maximal walking speed, grip strength, hanging endurance, daily food intake, and daily activity were all significantly higher in PLD mice than PL mice, while body weight changes were not statistically different (FIGS. 9B-9G). These results indicated that clearance of p21^{high} cells can alleviate physical frailty in old mice.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A transgenic non-human animal, the nucleated cells of which contain a transgene, said transgene comprising a p21 promoter sequence operably linked to a nucleic acid sequence encoding a Cre recombinase (Cre) polypeptide fused to a tamoxifen-inducible estrogen receptor (ER^{T2}) domain.

2. The transgenic non-human animal of claim 1, wherein said transgene further comprises, 3′ of said nucleic acid sequence encoding said Cre polypeptide fused to an ER^{T2} domain, an internal ribosome entry site (IRES) and a nucleotide sequence encoding a marker.

3. The transgenic non-human animal of claim 2, wherein said marker is a green fluorescent protein (GFP).

4. The transgenic non-human animal of claim 2, wherein one or more of visceral fat cells, brain cells, intestine cells, heart cells, liver cells, and skeletal muscle cells of said animal express said Cre polypeptide fused to an ER^{T2} domain and said marker.

5. The transgenic non-human animal of claim 1, wherein said non-human animal is a mouse.

6. The transgenic non-human animal of claim 1, wherein said p21 promoter sequence has at least 95% sequence identity with SEQ ID NO:1.

7. The transgenic non-human animal of claim 1, wherein said transgene is within an H11 genomic locus of said non-human animal.

8. The transgenic non-human animal of claim 1, wherein one or more of visceral fat cells, brain cells, intestine cells, heart cells, liver cells, and skeletal muscle cells of said animal express said Cre polypeptide fused to an ER^{T2} domain.

9. A nucleic acid comprising a p21 promoter sequence operably linked to a nucleotide sequence encoding a Cre polypeptide fused to an ER^{T2} domain.

10. The nucleic acid of claim 9, further comprising, 3′ of said nucleotide sequence encoding said Cre polypeptide fused to an ER^{T2} domain, an IRES and a nucleotide sequence encoding a marker.

11. The nucleic acid of claim 10, wherein said marker is a GFP.

12. The nucleic acid of claim 9, wherein said p21 promoter sequence has at least 95% sequence identity with SEQ ID NO:1.

13. The nucleic acid of claim 9, wherein said Cre polypeptide has an amino acid sequence at least 95% identical to SEQ ID NO:4.

14. The nucleic acid of claim 9, wherein said ER^{T2} domain has an amino acid sequence at least 95% identical to SEQ ID NO:6.
